(19) United States
(12) Patent Application Publication
(10) Pub. No.: US 2002/0147170 A1
(43) Pub. Date: Oct. 10, 2002

(54) Constitutively active, hypersensitive, and nonfunctional recepors as novel therapeutic agents

(76) Inventors: Alan S. Kopin, Wellesley, MA (US); Martin Beinborn, Boston, MA (US)

Correspondence Address: CLARK & ELBING LLP, 101 FEDERAL STREET, BOSTON, MA, 02110, US

(21) Appl. No.: 10/039,645

(22) Filed: Oct. 25, 2001

Related U.S. Application Data
(60) Provisional application No. 60/243,550, filed on Oct. 26, 2000.

(51) Int. Cl. A61K 48/00

(52) U.S. Cl. 514/44

(57) ABSTRACT

The invention features nucleic acids encoding constitutively active, hypersensitive, or nonfunctional receptors as novel therapeutic agents. The invention also features a method of treating a mammal, preventing a disease or disorder, or improve health by administering nucleic acids encoding constitutively active, hypersensitive, or nonfunctional receptors.

### CROSS REFERENCE TO RELATED APPLICATIONS

This application claims the benefit of the filing date of U.S. provisional application, U.S. Ser. No. 60/243,550, filed Oct. 26, 2000.

### STATEMENT AS TO FEDERALLY SPONSORED RESEARCH

This application was supported in part by NIH grant DK46767. The government may have certain rights to this invention.

### BACKGROUND OF THE INVENTION

In general, this invention relates to the use of nucleic acids encoding constitutively active, hypersensitive, or nonfunctional receptors in novel therapeutic compositions and methods.

A major focus of current scientific research is the identification of novel therapeutic agents that bind endogenous receptors (e.g., G protein-coupled receptors, single transmembrane receptors, and nuclear receptors). Particularly desirable agents are agonists and antagonists that activate or block endogenous receptors, respectively, to provide therapeutic benefit. However, such agonist or antagonist drug therapies may be difficult to find and often carry the risk of severe or undesirable side effects. An alternative to the use of agonist drug therapy is provided by the present invention in the form of nucleic acids encoding therapeutically effective constitutively active or hypersensitive receptors. Additionally, an alternative to the use of antagonist drug therapy is provided by the present invention in the form of nucleic acids encoding nonfunctional receptors.

### SUMMARY OF THE INVENTION

The present invention provides methods of treating or preventing a wide range of disorders by administering to a mammal a nucleic acid encoding a receptor having altered activity. These methods may be used to treat a disorder, prevent a disorder, or improve the health of a mammal. Such nucleic acids may encode receptors that are constitutively active, hypersensitive, or nonfunctional. According to the invention, such constitutively active, hypersensitive, or nonfunctional receptors may be G protein-coupled receptors, single transmembrane receptors, or nuclear receptors (for example, steroid hormone receptors).

One particularly preferred constitutively active receptor, which is also a hypersensitive receptor, is a mu opioid receptor. In one example, this receptor is constitutively active as a result of a single point mutation in transmembrane domain 3, preferably, an Asn to Ala point mutation at position 150 of the rat mu opioid receptor of SEQ ID NO: 1, or the human equivalent. Nucleic acids encoding consitutively active mu opioid receptors are useful therapeutic agents for the treatment of pain (for example, back pain) by administration of the nucleic acid, for example, to the intrathecal space of the spinal column. Such administration may block the sensory signal for pain en route to the brain so that pain at any particular location in the body is not perceived by the individual being treated.

Other preferred constitutively active receptors for administering to a mammal include constitutively active dopamine receptors, for example, dopamine 1 or dopamine 2 receptors. These receptors may be administered alone, in combination with one another, and/or in combination with other therapeutics, for the treatment of Parkinson's disease. Preferably, administration is to the brain (for example, to the striatum).

Nucleic acids encoding hypersensitive receptors may also be administered as therapeutic agents according to the invention. In addition to the mu opioid receptor described above, a hypersensitive erythropoietin receptor may also be utilized. This hypersensitive erythropoietin receptor may be used to treat or prevent anemia.

Alternatively, nonfunctional receptors may be administered therapeutically. For example, a nonfunctional CCK-BR receptor may be administered to treat or prevent peptic ulcer disease.

For any of the above methods, expression of the receptor may be accomplished using any promoter or vector system. If desired, receptors may be expressed under the control of an inducible, constitutive, or tissue specific promoter. Viral as well as non-viral vectors may be utilized, with retroviral, adenoviral, and adeno-associated viral vectors being preferred. Viral or nonviral vectors may include cell specific ligands that target administration to a specific cell type in the mammal.

Another integral feature of the present invention is the provision of therapeutic compositions including any of the nucleic acids described herein, such as any nucleic acid encoding a constitutively active, hypersensitive, or nonfunctional receptor (preferably, a constitutively active, hypersensitive, or nonfunctional receptor that is a G protein-coupled receptor, a single transmembrane receptor, or a nuclear receptor) admixed with a pharmaceutically acceptable carrier. One skilled in the art will appreciate that the therapeutic composition is preferably administered at a unit dose sufficient to reduce or eliminate the symptoms of a disease or disorder in a mammal, and such a dose can be easily determined by one of ordinary skill in the art.

The present invention further provides kits containing the nucleic acids and/or therapeutic compositions of the present invention for administration to an individual, for example, an individual diagnosed with a disease or a disorder. The kits may include all reagents required for facile administration by any known route to a patient suffering from a disease or disorder of which the symptoms may be reduced by expression in vivo of a receptor having altered activity. Such individuals may be individuals that have been identified as carriers of a particular polymorphism in a receptor that is linked to the occurrence of a disease, or individuals that may simply be suffering from an acute condition of which the symptoms may be reduced by the inventive approach. The nucleic acids are preferably in containers that also include a pharmaceutically acceptable carrier.

In yet another aspect, the gene therapeutic methods, compositions, and kits of the present invention may be used to improve the existing state of health of an individual, for example, by lengthening the individual's life span, improving the individual's physiology, improving the individual's cosmetic appearance, preventing aging (or the appearance of aging) of the individual, increasing the individual's strength, improving the individual's memory, or improving athletic ability of the individual etc. Additional health improving uses will be apparent to those skilled in the art.

By a “constitutively active receptor” is meant a receptor with a higher basal activity level than the corresponding wild-type receptor or a receptor possessing the ability to spontaneously signal in the absence of activation by a positive agonist. This term includes wild-type receptors that are naturally constitutively active (e.g., naturally occurring receptors, including naturally occurring polymorphic receptors and wild-type receptors) and that have a higher basal activity level than a corresponding vector lacking a gene encoding a receptor. The term also includes receptors having mutations (for example, point mutations), as well as receptor chimeras and fusion proteins. In addition, a receptor may be made constitutively active by co-expression with a second protein (such as a homer protein) that regulates receptor activity. The constitutive activity of a receptor may be established by comparing the basal level of signaling, such as second messenger signaling, of a mutant receptor to the basal level of signaling of the wild-type receptor. A constitutively active receptor exhibits at least a 5% increase in basal activity, preferably, at least a 25% increase in basal activity, more preferably at least a 50% increase in basal level activity. It is common for a constitutively active receptor, e.g., a polymorphic constitutively active receptor that is associated with a disease phenotype, to display a relatively small increase in constitutive activity. Preferably, the basal activity of a constitutively active receptor can be confirmed by its decrease in the presence of an inverse agonist.

“Basal” activity means the level of activity (e.g., activation of a specific biochemical pathway or second messenger signaling event) of a receptor in the absence of stimulation with a receptor-specific ligand (e.g., a positive agonist). Preferably, the basal activity is less than the level of ligand-stimulated activity of a wild-type receptor. However, in certain cases, a mutant receptor with increased basal activity might display a level of signaling that approximates, is equal to, or even exceeds the level of ligand-stimulated activity of the corresponding wild-type receptor.

By a “hypersensitive receptor” is meant a receptor having the ability to amplify the input of an endogenous ligand (e.g., a positive or negative agonist), as compared to the wild-type receptor. Such receptors deliver an increased receptor-induced signal in response to a ligand compared to a corresponding wild-type receptor. Hypersensitive receptors may include mutations (for example, point mutations), or may be constructed, for example, as receptor chimeras or fusion proteins. A hypersensitive receptor exhibits at least a 5% increase, preferably, at least a 25% increase, and more preferably at least a 50% increase in ligand-stimulated activity as compared to a corresponding wild-type receptor.

By a “nonfunctional” receptor is meant a receptor that has decreased signaling in response to ligand binding. A nonfunctional receptor may also be a receptor that has reduced binding to a ligand and thus may transmit a weakened signal in response to ligand stimulation. However, ligand binding may not necessarily occur in every type of nonfunctional receptor. The nonfunctional receptor may be a receptor that is deficient in ligand binding. According to the invention, any mutation that reduces or eliminates ligand-stimulated signaling of a receptor qualifies as a nonfunctional receptor. For example, a nonfunctional receptor could be a receptor that does not bind ligand, and therefore does not transmit a signal in response to ligand binding. A nonfunctional receptor exhibits at least a 5% decrease in ligand-stimulated signal transduction, preferably, at least a 25% decrease in ligand-stimulated signal transduction, and more preferably, at least a 50% reduction in ligand-stimulated signal transduction. Receptors having a decrease in receptor signaling, but not a complete loss of receptor signaling, may be referred to as “hyposensitive receptors.” Such a hyposensitive receptor may have the characteristics of a partial antagonist in the cell and therefore be used in place of partial antagonistic drug therapy treatments. Nonfunctional or hyposensitive receptors may include mutations (for example, point mutations). These receptors may also be constructed as chimeric receptors or fusion proteins.

A “naturally-occurring” receptor refers to a form or sequence of a receptor as it exists in an animal, or to a form of the receptor that is homologous to the sequence known to those skilled in the art as the “wild-type” sequence. Those skilled in the art will understand “wild type” receptor to refer to the conventionally accepted “wild-type” amino acid consensus sequence of the receptor, or to a “naturally-occurring” receptor with normal physiological patterns of ligand binding and signaling.

A “mutant receptor” is understood to be a form of the receptor in which one or more amino acid residues in the predominant receptor occurring in nature, e.g., a naturally-occurring wild-type receptor, have been either deleted or replaced. Alternatively additional amino acid residues have been inserted.

By “mu opioid receptor” is meant a polypeptide having the analgesic characteristics of the mu opioid receptor, or other associated mu opioid receptor biological activities. These activities include, for example, high affinities for analgesic and addicting opiate drugs (e.g., morphine and fentanyl) and opioid peptides (e.g., enkephalins, endorphins, and dynorphins (Rothman et al., Synapse 21:60-64 (1995); Wang et al., Proc. Natl. Acad. Sci. USA 90:10230-10234 (1993); Li et al., J. Mol. Evol. 43:179-184 (1996)). In particular examples, the mu opioid receptor has nanomolar affinities for morphine and the enkephalin analog DADLE and clear recognition of naloxonazine (Wang et al., supra; Wolozin et al., Proc. Natl. Acad. Sci. USA 78:6181-6185 (1981); Eppier et al., J. Biol. Chem. 268(35):26447-26451; Golstein et al., Mol. Pharmacol. 36:265-272 (1989)). Ligand binding initiates coupling of the mu opioid receptor to adenylate cyclase, causing a decrease in adenylate cyclase activity and a corresponding decrease in the level of intracellular cAMP (Wang et al., supra).

By “substantially pure nucleic acid” is meant nucleic acid (e.g., DNA or RNA) that is free of the genes, which, in the naturally-occurring genome of the organism from which the DNA of the invention is derived, flank the gene. The term therefore includes, for example, a recombinant DNA which is incorporated into a vector; into an autonomously replicating plasmid or virus; or into the genomic DNA of a prokaryote or eukaryote; or which exists as a separate molecule (e.g., a cDNA or a genomic or cDNA fragment produced by PCR or restriction endonuclease digestion) independent of other sequences. It also includes a recombinant DNA, which is part of a hybrid gene encoding additional polypeptide sequence.

“Transformed cell” means a cell into which (or into an ancestor of which) has been introduced, by means of recombinant DNA techniques, a DNA molecule encoding (as used herein) a polypeptide described herein (for example, a mu opioid receptor polypeptide).

“Promoter” means a minimal sequence sufficient to direct transcription. Also included in the invention are those promoter elements which are sufficient to render promoter-dependent gene expression controllable for cell-type specific, tissue-specific, or inducible expression by external signals or agents; such elements may be located in the 5′ or 3′ regions of the native gene. A promoter element may be positioned for expression if it is positioned adjacent to a DNA sequence so it can direct transcription of the sequence.

“Operably linked” means that a gene and a regulatory sequence(s) are connected in such a way as to permit gene expression when the appropriate molecules (e.g., transcriptional activator proteins) are bound to the regulatory sequence(s).

“Reporter assay system” means any combination of vectors typically used for measuring transcriptional activation. A typical reporter assay system includes at least a reporter construct and an expression vector encoding the polypeptide that activates (e.g., directly) or causes to activate (e.g., indirectly) expression of the reporter construct. The reporter assay system may also include additional expression vectors encoding other polypeptides that participate in activation of the reporter construct.

“Expression vectors” contain at least a promoter operably linked to the gene to be expressed.

A “reporter construct” includes at least a promoter operably linked to a reporter gene. Such reporter genes may be detected directly (e.g., by visual inspection) or indirectly (e.g., by binding of an antibody to the reporter gene product or by reporter product-mediated induction of a second gene product). Examples of standard reporter genes include genes encoding the luciferase, green fluorescent protein, or chloramphenicol acetyl transferase gene polypeptides (see, for example, Sambrook, J. et al., Molecular Cloning: a Laboratory Manual, Cold Spring Harbor Press, N.Y., or Ausubel et al., Current Protocols in Molecular Biology, Greene Publishing Associates, New York, N.Y., V 1-3, 2000, incorporated herein by reference). Expression of the reporter gene is detectable by use of an assay that directly or indirectly measures the activity of the polypeptide encoded by the reporter gene. Preferred reporter constructs also include a response element.

A “response element” is a nucleic acid sequence that is sensitive to a particular signaling pathway, e.g., a second messenger signaling pathway, and assists in driving transcription of the reporter gene in cooperation with the promoter. As used herein, “response element” may also refer to a promoter that is activated in response to signaling through a particular receptor.

By “disease” or “disorder” is meant any ailment or adverse condition that can be diagnosed in a mammal. As used herein, disease or disorder can be used to refer to a physical symptom such as a pain or an ache (e.g., chronic back pain or arthritis etc.) or to refer to a severe condition, such as cancer.

“Disease-inhibiting amount” or “disorder-inhibiting amount” means an amount of nucleic acid that, when delivered to a cell, tissue, or site in vivo or ex vivo, is capable of reducing, delaying, or stabilizing the symptoms or progression of a disease or disorder with which a patient has been diagnosed. For example, one particularly preferred disease or disorder to be treated by the invention is pain, particularly back pain. According to one preferred embodiment of the invention, an amount of nucleic acid, or a “pain inhibiting amount” of nucleic acid, is preferably delivered to the intrathecal space sufficient to reduce pain at that site.

By “improvement of health” is meant a change of the normal (average) state of health to a state of heath that is superior to the normal state of health (e.g., increased strength, prevention of aging, improved memory, or improved athletic ability).

As used herein, “second messenger signaling activity” refers to production of an intracellular stimulus (including, but not limited to, cAMP, cGMP, ppGpp, inositol phosphate, calcium ion) in response to activation of the receptor, or to activation of a protein in response to receptor activation, including but not limited to a kinase, a phosphatase, adenylate cyclase, or phohpholipase C, or to activation or inhibition of a membrane channel.

### BRIEF DESCRIPTION OF THE DRAWING

FIG. 1 is a table of constitutively active Class A G protein-coupled receptors (SEQ ID NOS: 2-70). The mutations that impart constitutive activity to the receptors are indicated.

FIG. 2 is a graph showing the constitutive activity of a D146M MC-4 receptor mutant as assayed by measuring basal level cAMP production.

FIG. 3 is a graph showing the constitutive activity of the L325E CCK-BR receptor as assayed using a luciferase reporter assay.

FIG. 4 is a graph showing the constitutive activity of the Asn150Ala rat mu opioid receptor as assayed using a luciferase reporter assay. This is evidenced by the following: (1) agonist (DAMGO) stimulation of the receptor leads to a decrease in forskolin induced activity, indicating that the receptor works through an inhibiting pathway; (2) forskolin induced activity in the absence of DAMGO is lower with coexpression of mutant receptor (vs. wild-type receptor), indicating ligand independent activity of the inhibitory pathway.

FIG. 5 is a graph showing the effects of forskolin stimulation on HEK293 cells transfected with pcDNA1 and a CRE-Luc reporter construct.

FIG. 6 is a graph showing the sensitivity of the reporter constructs, SMS-luc, SRE-Luc, and SRE-Luc+Gq5i to ligand-mediated activation of the mu opioid receptor.

FIG. 7 is a graph showing the constitutive activity of the Asn150Ala rat mu opioid receptor as assayed using the SRE-Luc/Gq5i luciferase reporter assay.

FIG. 8 is an illustration of a seven transmembrane domain Class A G protein-coupled receptor. (Selected residues are indicated.)

FIG. 9 is an illustration showing the highly conserved “N” residue among the mu opioid receptor, the bradykinin B2 receptor, and the angiotensin II AT1A receptor. In each of these receptors, mutation of the “N” residue leads to constitutive activity.

FIG. 10 is an illustration showing the “DRY” motif, which is highly conserved among the oxytocin, vasopressin-V2, cholecystokinin-A, melanocortin-4, and 1b adrenergic receptors. In addition, mutation of this “DRY” motif in these receptors leads to constitutive activity.

FIG. 11 is a graph showing the constitutive activity of the D146M MC-4 receptor as assayed using a luciferase reporter assay.

FIG. 12 is an illustration showing the −13 and −20 positions relative to the “CWLP motif.” Mutation in the −13 position in the 1A adrenergic receptor, the α2C adrenergic receptor, the β2 adrenergic receptor, the serotonin 2A receptor, the cholecystokinin-B receptor, the platelet activating factor receptor, and the thyroid stimulating hormone receptor leads to constitutive activity.

FIG. 13 is an illustration showing a sequence alignment of the human kappa opioid receptor (ork), the rat kappa opioid receptor (orkr), the human mu opioid receptor (orm), the rat mu opioid receptor (ormr), the human delta opioid receptor (ord), the rat type 1A angiotensin II receptor (AT1A), and the human bradykinin receptor (B2) (SEQ ID NOS: 71-77). Also shown is the N residue, which is located 14 amino acids to the amino-terminus of the “DRY” motif (−14).

FIG. 14 is an illustration showing the amino acid sequence (top to bottom) of the mouse mu opioid receptor, the rat mu opioid receptor, the bovine mu opioid receptor, the human mu opioid receptor, the pig mu opioid receptor, the white sucker (ws) opioid receptor, the angiotensin AT-1 receptor, and the bradykinin-B2 receptor. The N position is highlighted (−14 from the DRY motif). Mutation of this residue leads to constitutive activity in each of these receptors.

FIG. 15 is a graph showing the hypersensitivity of the Asn150Ala rat mutant mu opioid receptor (•), which is also constitutively active, compared to the wild-type mu opioid receptor (∇). Ligand (Damgo) was titrated onto the cells expressing either the mutant or the wild-type mu opioid receptor and the luciferase activity was measured to assess the sensitivity of the receptor to ligand stimulation.

FIG. 16 is a graph showing that mutation of the Val at position 331 of the CCK-BR gastrin receptor to a Glu dramatically reduces ligand-stimulated activation of the receptor. CCK-BR activity was determined by measuring ligand induced inositol phosphate production. The illustration to the left of the graph shows the seven membrane spanning topology of the CCK-BR receptor. The larger shaded circle shows amino acid 331.

FIG. 17 is a map of a shuttle vector for adenovirus (pACCMV.pLpA).

### DESCRIPTION OF THE PREFERRED EMBODIMENTS

The present invention is based on the recognition that nucleic acids encoding constitutively active, hypersensitive, or nonfunctional receptors can be used as therapeutic agents. According to the present invention, constitutively active, hypersensitive, or nonfunctional receptors include constitutively active, hypersensitive, or nonfunctional G protein-coupled receptors (e.g., opiate receptors), single transmembrane domain receptors (e.g., the erythropoietin receptor (EPOR)), nuclear receptors (e.g., steroid hormone receptors, such as the estrogen receptor), and soluble receptors (Appendices A-E are lists of known receptors that classify as G protein-coupled receptors, single transmembrane domain receptors, and nuclear receptors). In certain preferred embodiments, the invention provides methods of identifying nucleic acids encoding constitutively active, hypersensitive, and nonfunctional receptors. In yet other preferred embodiments, the invention provides a method of treating or preventing a disease in a mammal by administering to the mammal a nucleic acid encoding a constitutively active, hypersensitive, or nonfunctional receptor. Alternatively, a nucleic acid encoding a constitutively active, hypersensitive, or nonfunctional receptor provides a means of improving the physiology or existing state of health of a mammal (e.g., increase life span, cosmetic appearance, prevent aging, increase strength, improve memory, improve athletic ability, etc). For example, a constitutively active or hypersensitive erythropoietin receptor has been shown to improve the physiology of a person such that the person has outstanding athletic ability, e.g., improved stamina (Watowich et al., Blood 94(7):2530-2532 (1999); Yoshimura et al., Oncologist 1(5):337-339 (1996)).

Those skilled in the art will appreciate that many aspects of the invention that apply to constitutively active receptors also apply to hypersensitive and nonfunctional receptors. For example, one skilled in the art will recognize that many of the assays described herein may be used to measure constitutive basal activity or ligand-stimulated hypersensitive activity. Alternatively, the skilled artisan will appreciate that any assay typically used to measure a ligand-stimulated receptor response can be used to detect the absence of that response in a non-functional receptor. In addition, any of the gene therapy methods provided herein may be applied to nucleic acids encoding either constitutively active, hypersensitive, or non-functional receptors.

A key feature of the present invention is that it provides a valuable alternative to the administration of agonist and antagonist drugs for the treatment of disease. In contrast to agonist drug therapy, which enhances the activity of endogenous receptors, the present invention provides a recombinant constitutively active receptor that delivers a constitutive intracellular signal that is frequently less than or equivalent to, or perhaps greater than, the signal generated by the agonist drug. Similarly, a recombinant hypersensitive receptor may be used to deliver an enhanced ligand-stimulated signal intracellularly. In addition, in contrast to antagonist drug therapy, which reduces or inhibits the activity of endogenous receptors, the present invention provides recombinant non functional receptors that act as a sink for the endongenous ligand, yet do not transduce a ligand-stimulated signal. Unlike conventional agonist drug therapy, the inventive treatment may be generally both safe and effective, e.g., may induce an intracellular signal sufficient to mimic agonist or antagonist without any side effects.

With respect to the constitutively active receptors, without being bound to any particular theory, the increased basal level activity of the constitutively active receptor is likely due to increased ligand-independent receptor signaling. For example, the expressed receptor may assemble intracellularly and constitutively activate a specific second messenger signaling pathway. Any therapeutic benefit achieved by constitutive second messenger signaling through a recombinant constitutively active receptor provides a number of advantages over systemic administration of agonist drugs, including the elimination of the need for a strict, daily administration regime. For example, the benefit of obtaining a steady state level of signaling, without having to compensate for the normal metabolic half life of an agonist, is inherent within the system. It is important to appreciate that, according to the present invention, even low level constitutive activity can have beneficial therapeutic effects.

With respect to the hypersensitive receptors, we propose, without limitation, that the increased sensitivity to ligand stimulation of a hypersensitive receptor is likely due to an increased affinity of the ligand for the receptor. This increased affinity is then reflected in an increased potency of the receptor upon ligand binding (i.e., the signal generated by ligand binding is amplified compared to the wild-type level of signaling).

Similarly, with respect to the nonfunctional receptors, without limiting the mechanism of the invention, it is likely that the inhibition of a signal that is contributing to disease or reduced health, results in a reduction in disease symptoms or improvement in health. For example, the administration of a nonfunctional receptor, i.e., a dominant negative mutant of the receptor, may bind the ligand for the receptor but lack the signaling function of the receptor. This would effectively reduce the extracellular concentration of the ligand for the receptor, while eliminating the signal generated by the particular receptor that is contributing to disease or reduced health.

According to the present invention, constitutively active receptors include naturally occurring constitutively active receptors and non-naturally occurring (i.e., mutant) constitutively active receptors. The present invention provides methods of identifying both naturally and non-naturally occurring constitutively active receptors. According to the present invention, constitutively active receptors with increased basal activity are compared to the appropriate negative control. For example, naturally occurring constitutively active receptors can be identified by exhibiting an increased basal level of signaling compared to the activity of a vector lacking a gene encoding a receptor. Alternatively, mutant receptors having constitutive activity can be identified by comparing the basal level of signaling of the mutant constitutively active receptor to the basal level of signaling of the wild-type receptor. An increase (e.g., by at least 5%) in basal level activity in a candidate receptor compared to a control or wild-type receptor identifies a constitutively active receptor.

Many naturally occurring and non-naturally occurring constitutively active receptors have been previously identified and are available in the art. As described herein, this information can be harnessed and used as a tool to identify additional constitutively active receptors. According to the present invention, the amino acid and/or nucleic acid sequences of known constitutively active receptors are assembled into a database, which is used to identify conserved domains that are important for constitutive activity or mutations within those domains that impart constitutive activity onto the receptor. The sequences of constitutively active polypeptides (mutant and wild-types) in such a database are then compared to the sequence of a given non-constitutively active receptor, and conserved domains are identified between the nonconstitutively active receptor and the constitutively active receptors. This information is further used to identify specific residues within a given nonconstitutively active (e.g., wild-type) receptor that are likely to impart constitutive activity to the nonconstitutively active receptor upon mutation.

Once specific positions in a given nonconstitutively active receptor are targeted for mutation, receptors containing the identified mutations are generated using routine methods and screened for increased constitutive activity (see, for example, Sambrook, J. et al., Molecular Cloning: a Laboratory Manual, Cold Spring Harbor Press, N.Y., or Ausubel et al., Current Protocols in Molecular Biology, Greene Publishing Associates, New York, N.Y., V 1&3, 2000, incorporated herein by reference). Preferably, an increase in basal level activity is detected by measuring an increase in basal level signaling in the mutant receptor, compared to the wild-type receptor. The skilled artisan will appreciate that any assay typically used for measuring the ligand-stimulated activity of the wild-type receptor may also be used to measure the basal level activity of a mutant receptor. Such assays are discussed in further detail herein, below.

These general principles can be easily applied by one of ordinary skill in the art to identify hypersensitive receptors or nonfunctional receptors. Hyper-sensitive receptors are receptors that deliver an increased receptor induced signal in response to a ligand, compared to the wild-type receptor. In preferred embodiments, non-naturally occurring receptors that are hypersensitive are identified by comparing the ligand-induced activity of the wild-type receptor to the ligand-induced activity of the mutant receptor; a hypersensitive receptor being identified by its ability to display a stronger signal to a given concentration of ligand than the wild-type receptor. For example, if 5 μM ligand induces a 5-fold stimulation of activity in a wild-type receptor, compared to a negative control, 5 μM ligand may stimulate a 10-fold stimulation in activity in a hypersensitive receptor, compared to the same negative control. Indeed, hypersensitive mutants of the EPO receptor and the mu opioid receptor have already been identified (Watowich et al., Blood 94(7):2530-2532 (1999), incorporated herein by reference). Specifically, mutations in the EPOR that result in familial erythrocytosis result from premature termination of the receptor cytoplasmic region. EPOR mutants lacking the cytoplasmic tail region do not undergo tyrosine phosphorylation, allowing JAK2 activation to continue for a longer period of time, and thus the signal is generated more efficiently (Watowich et al., supra; Yoshimura et al., Oncologist 1(5):337-339 (1996); Tilbrook et al., Int. J. Biochem. Cell Biol. 31(10):1001-1005 (1999); de la Chapelle et al., Proc. Natl. Acad. Sci. USA 90(10):4495-4499 (1993); Kirby et al., Cytokines Cell Mol. Ther. 5(2):97-104 (1999); Yoshimura et al., Curr. Opin. Hematol. 5(3):171-176 (1998); Pharr et al., Proc. Natl. Acad. Sci. USA 90:938-942 (1993), incorporated herein by reference). According to one particularly preferred embodiment, a nucleic acid encoding a hypersensitive EPOR is used as a gene therapeutic reagent to treat anemia. Alternatively, hypersensitive EPO receptors may be used to improve the athletic potential. Nonfunctional receptors can be similarly generated and tested for an absence of ligand stimulated response compared to the functional wild-type receptor. Such nonfunctional receptors may be used as treatments for polycythemia vera.

The present invention further provides a method of treating a mammal, preferably, a human, diagnosed with a particular disease, by administering a nucleic acid encoding a constitutively active, hypersensitive, or nonfunctional receptor. In preferred embodiments, the nucleic acids of the invention are delivered to specific cells, tissues, or sites in a mammal suffering from a disease. In particularly preferred embodiments, the nucleic acids of the invention are delivered in vivo to a specific site in the body. For example, the nucleic acids of the present invention may be administered (e.g., by injection) directly into a tumor for treatment of cancer. Alternatively, the nucleic acids of the invention may be administered to a particular diseased organ, for example, the liver or kidney. As but another example, the nucleic acids may be delivered to a patient experiencing pain. Most preferably, therapeutic nucleic acids encoding constitutively active, hypersensitive, or nonfunctional receptors are administered to a site at which a therapeutic benefit will be achieved. These sites include surfaces, such as skin, mucosal surfaces (e.g., in bronchial/nasal passages or genitourinary tract). Indeed, administration of nucleic acids to any bodily surface is particularly desirable. Typically, the inventive nucleic acid is delivered to the cells of a mammal and expressed by those cells to produce a polypeptide that spontaneously assembles into a supermolecular structure in vivo (e.g., in the lipid bilayer of a cell) and functions as a constitutively active, hypersensitive, or nonfunctional receptor.

In a related aspect, the present invention provides cells (in vivo or in vitro) containing substantially pure nucleic acids encoding a constitutively active, hypersensitive, or nonfunctional receptor of the invention. In one preferred embodiment, the cells are transfected with the nucleic acid in vitro and transferred to a patient in vivo to achieve therapeutic benefit. Such methods of ex vivo gene therapy are described in detail below. One example of a receptor that is amenable to such an approach is the human EPO receptor. A hypersensitive EPO receptor may be identified, such as the EPO receptor identified by Watowich et al. (supra), and transfected into human erythroid progenitor cells or bone marrow cells in vitro. The cells are then transferred to a patient diagnosed with anemia.

In yet another preferred embodiment, nucleic acids encoding constitutively active, hypersensitive, or nonfunctional receptors are coadministered with an agonist or antagonist to the receptor in order to treat a mammal having a disease. Alternatively, an agonist or antagonist is administered subsequent to the administration of the nucleic acid. Such treatment is particularly desirable if either the nucleic acid or the agonist alone are insufficient to achieve therapeutic benefit.

A wide variety of in vivo, in vitro, and ex vivo nucleic acid delivery systems for administration of constitutively active, hypersensitive, or nonfunctional receptors are available in the art. One particularly preferred nucleic acid delivery system is the viral vector delivery system. Viral vectors are particularly useful for in vivo gene therapy. Alternatively, a wide variety of non-viral nucleic acid delivery systems are available in the art. Such delivery systems are described in detail below.

In one preferred embodiment, a nucleic acid encoding any naturally constitutively active receptor (e.g., a wild-type receptor having constitutive activity) or any receptor having a mutation in its amino acid sequence that induces a higher basal activity than the corresponding wild-type receptor may be administered to a mammal to achieve therapeutic benefit. In another preferred embodiment, a nucleic acid encoding any receptor, e.g., a wild-type or mutant receptor, exhibiting hypersensitivity to a ligand may be administered to a mammal for the treatment of a particular disease. In yet another preferred embodiment, a nucleic acid encoding a nonfunctional receptor is administered to a mammal for treatment of a particular disease or condition. Alternatively, the nucleic acid may be administered with the goal of improving the state of health in the mammal.

For example, clinically useful constitutively active receptors include GLP-1 receptors for diabetes, somatostatin receptors for cancer, EPO receptors for anemia, estrogen receptors for menopause, melanocortin receptors for obesity, β2 adrenergic receptors for asthma etc. Similarly, examples of hypersensitive receptors that may be used in the present invention include the EPO receptors for anemia, mu opioid receptors for pain, estrogen receptors for menopause, melanocortin receptors for obesity, and β2 adrenergic receptors for asthma.

A nucleic acid encoding a nonfunctional receptor that may be administered to a mammal for treatment of a particular disease includes, for example, a nonfunctional CCK-BR receptor for treatment of peptic ulcer disease, a nonfunctional growth factor receptor for treatment of cancer, a nonfunctional estrogen receptor as an alternative to the treatment of cancer with tamaxofen (e.g., replacing the effect of an estrogen receptor antagonist), a nonfunctional erythropoietin receptor for treatment of polycythemia vera, a nonfunctional cytokine receptor as an anti-inflammatory, or a nonfunctional CCR-3 receptor for treatment of asthma.

In certain preferred embodiments of the invention, it may be desirable to target a recombinant nucleic acid to a specific cell type or tissue in vivo. It will be appreciated by one of ordinary skill in the art that the viral and non-viral vectors of the invention may include, or encode for the purpose of expression, one or more cell-, tissue-, or organ-specific ligands (e.g., a protein or polypeptide) for the purpose of targeting the nucleic acid to any cell-type in the body. Preferably, the one or more cell-, tissue, or organ-specific ligands are presented on the outside surface of the viral or non-viral vector. The ligand functions to target the vector to a specific tissue in vivo via its affinity for a particular molecule expressed on the surface of the target cell. Alternatively, the ligand may be an antibody directed to a particular cellular protein, preferably a cellular protein expressed on the surface of a cell. As noted above, the specificity of the vector can be changed by simply changing the polypeptide or antibody ligand that is responsible for targeting.

Thus, in one preferred embodiment, the invention provides viral and non-viral vectors encoding constitutively active, hypersensitive, or nonfunctional receptors capable of targeting the receptor to a specific cell type in the body. In other preferred embodiments, targeting is accomplished by direct administration (e.g., by injection) of nucleic acid encoding a constitutively active, hypersensitive, or nonfunctional receptor to a cell, tissue, organ, or site of interest. Of course one skilled in the art will appreciate that the cell, tissue, or organ to which the vector is targeted can be altered by simply changing the cell-specific ligand on the vector.

In other preferred embodiments, it may be desirable to titrate the activity of the constitutively active or hypersensitive receptor of the invention, i.e., to decrease or reduce the level of signaling. Alternatively, the level of nonfunctional receptor expressed in a cell may need to be controlled or altered, for example, to increase or decrease the inhibitory effect of the nonfunctional receptor. In order to achieve this result, the constitutively active, hypersensitive, or nonfunctional receptor is expressed under the control of an inducible promoter (e.g., the tetracycline inducible promoter). Expression from the inducible promoter is regulated by a benign small molecule (e.g., tetracycline). Expression is increased or decreased by controlling the amount of the small molecule administered, or expression is turned on or off by addition or removal of the small molecule, respectively. Alternatively, it may be desirable to use a constitutive promoter to maintain a constant level of expression of the constitutively active receptor. In yet another preferred embodiment, a tissue specific promoter may be used to target expression of a constitutively active, hypersensitive, or nonfunctional receptor to a particular tissue (see, for example, Gopalkrishnan et al., Nucleic Acids Res. 27(24):4775-4782 (1999); Huang et al., Mol. Med. 5(2):129-137 (1999)). Other inducible systems are widely available, e.g., the ecdysone inducible system (No et al., Proc. Natl. Acad. Sci, USA, 93(8):3346-3351, (1996); Invitrogen, Carlsbad, Calif.).

Identifying Constitutively Active Receptors

The present invention provides a method of identifying constitutively active, hypersensitive, or nonfunctional receptors and nucleic acids encoding constitutively active, hypersensitive, or nonfunctional receptors. Regarding constitutively active receptors, as described above, some receptors (e.g., wild-type receptors) are naturally constitutively active. Such naturally occurring constitutively active receptors are identified by simply comparing the basal activity of the wild-type receptor to that of a negative control. A suitable negative control is, for example, a cell lacking expression of the natural wild type receptor (e.g., a cell transfected with an empty expression vector, a cell transfected with a wild-type vector, or a cell transfected with a different receptor that has been previously established to lack constitutive activity (preferably both an empty expression vector and a non-constitutively active, wild-type vector are used)).

Alternatively, the present invention provides a method of identifying mutation-induced constitutively active, hypersensitive, or nonfunctional receptors. Preferably, the mutation-induced constitutively active, hypersensitive, or nonfunctional receptors are receptors of therapeutic interest. According to the present invention, mutation-induced receptors may be identified systematically by 1) identifying regions of homology between a wild-type receptor (e.g., a nonconstitutively active, nonhypersensitive, or functional receptor) and one or more receptors with the preferred activity (i.e., constitutively active, hypersensitive, or nonfunctional receptors); 2) introducing mutations into one or more regions of the wild-type receptor based on the identified region(s) of homology; and 3) assaying the mutant receptors for constitutive, hypersensitive, or nonfunctional activity. Methods of achieving each of these steps are described in detail below.

One skilled in the art will appreciate that the mutations can also be introduced by any random mutagenesis procedure standard in the art. A large variety of random mutagenesis kits are in fact commercially available. Once identified, e.g., in a yeast expression system, the constitutive, hypersensitive, or nonfunctional activity of the receptor may be confirmed, for example, using a mammalian expression system. Alternatively, screening can be directly performed in a mammalian cell expression system.

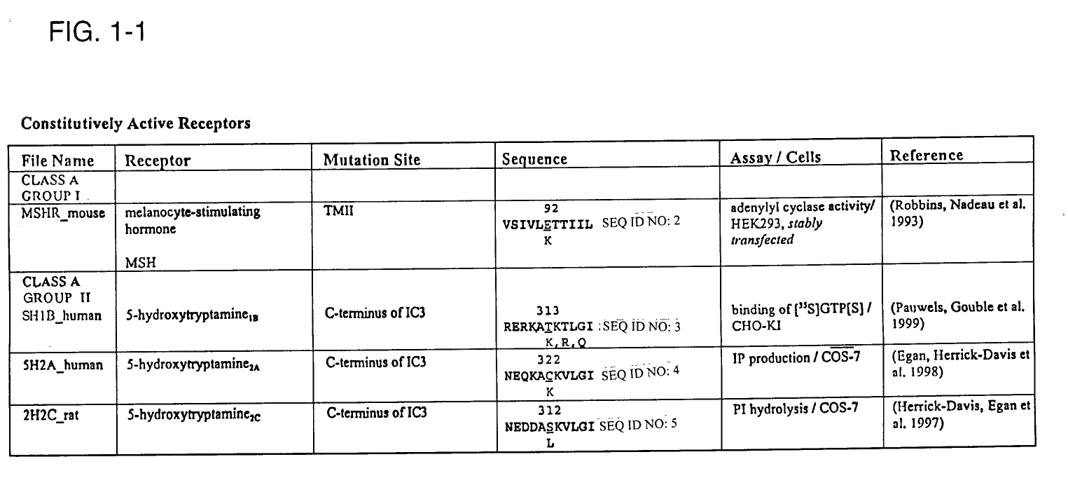

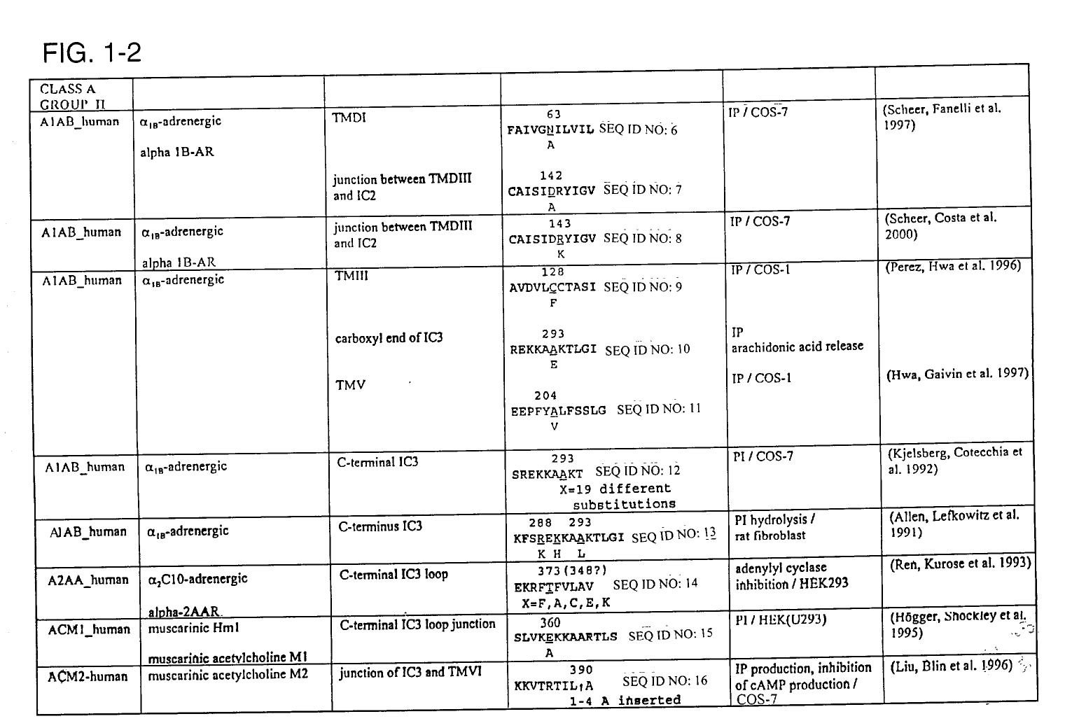

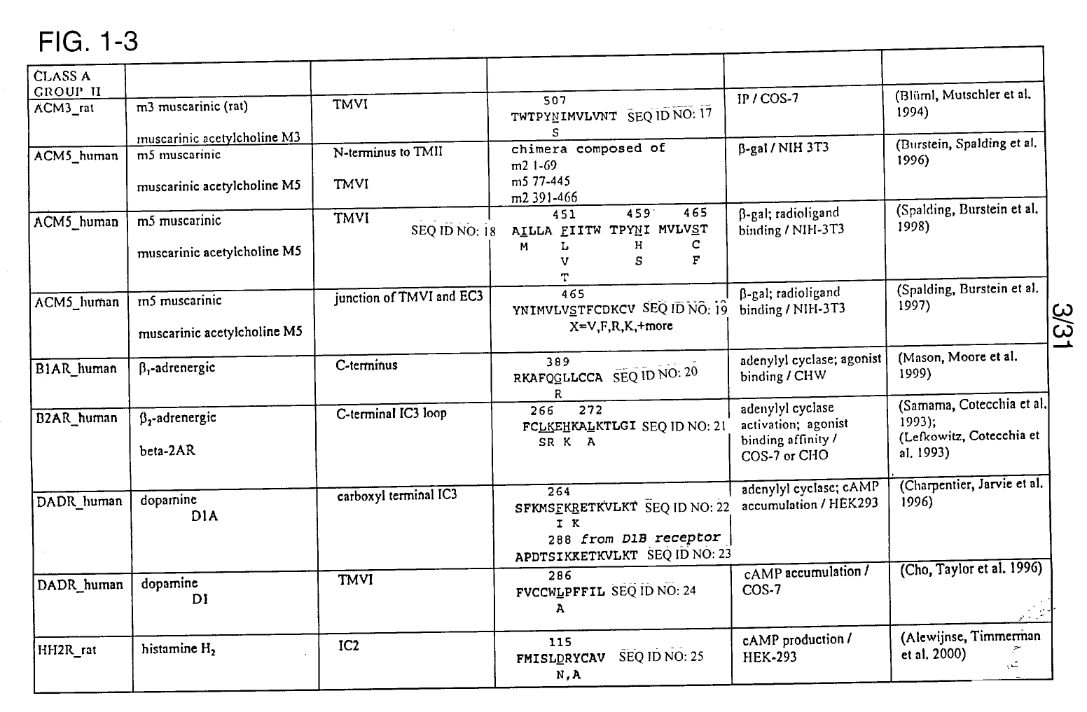

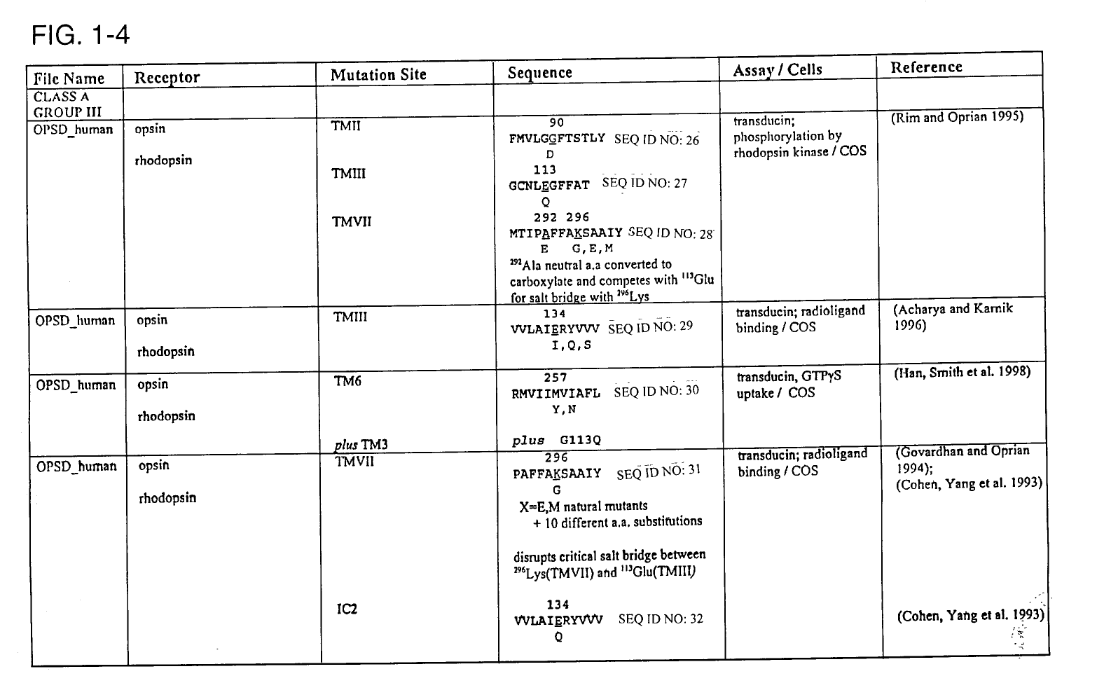

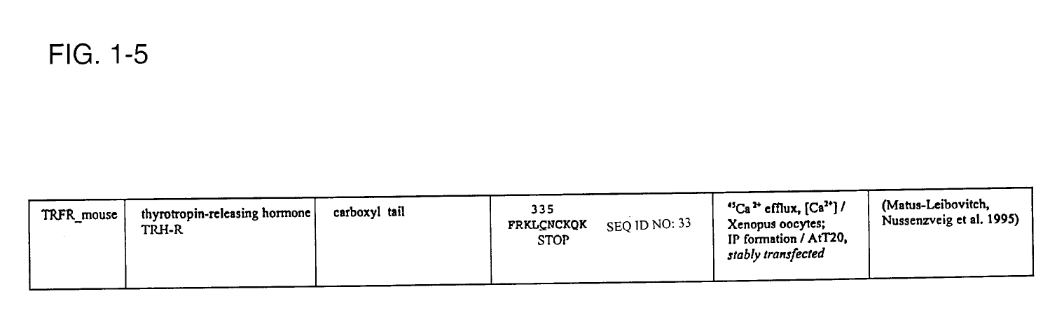

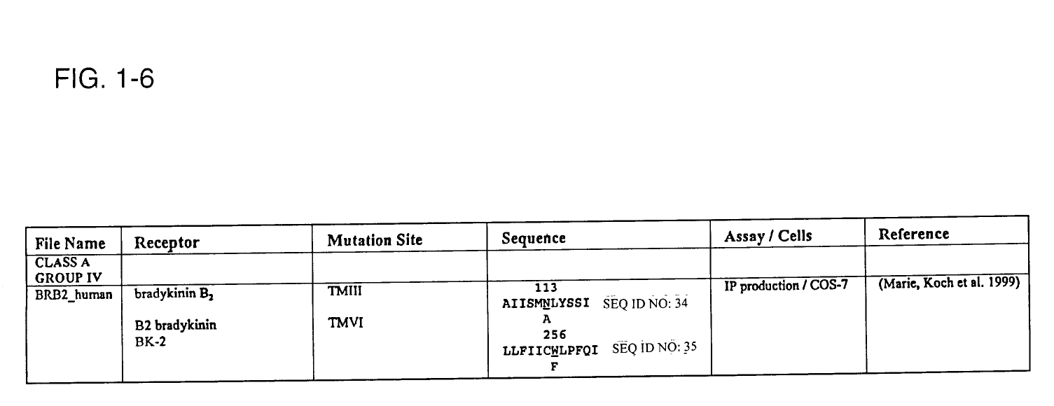

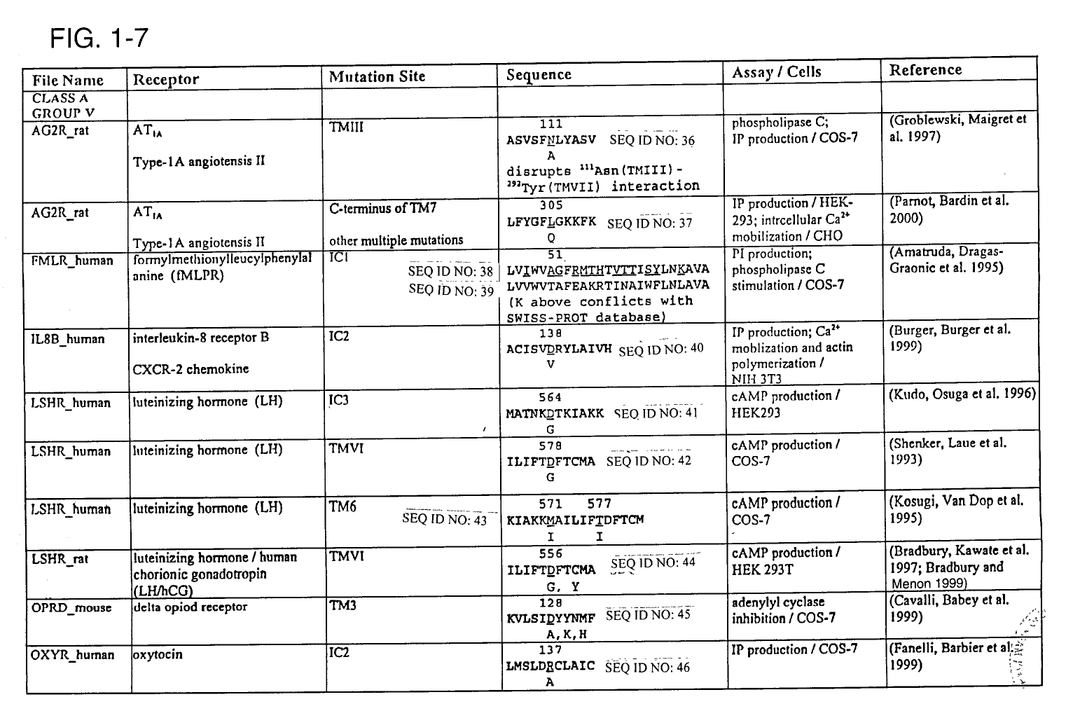

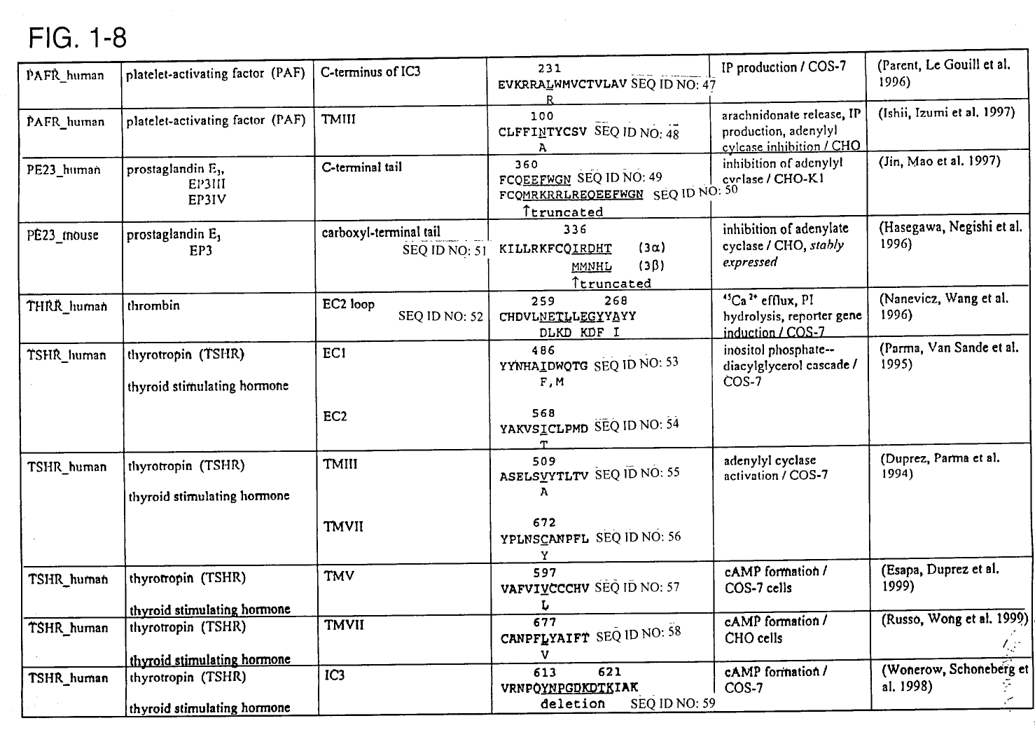

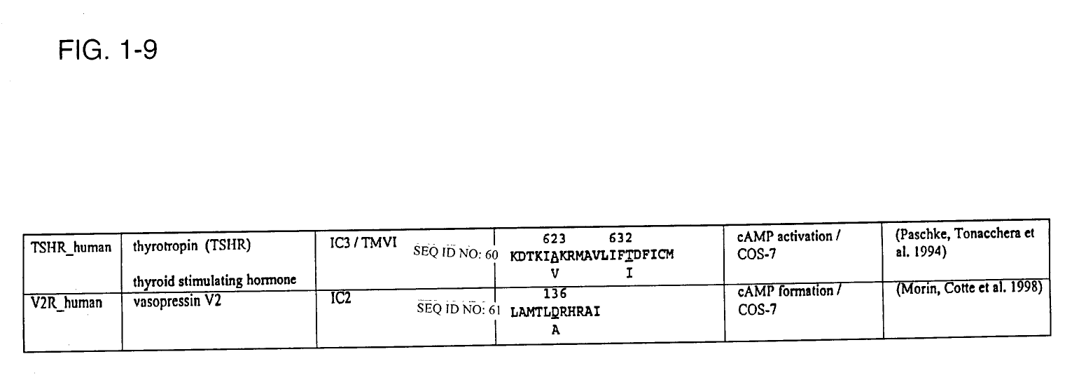

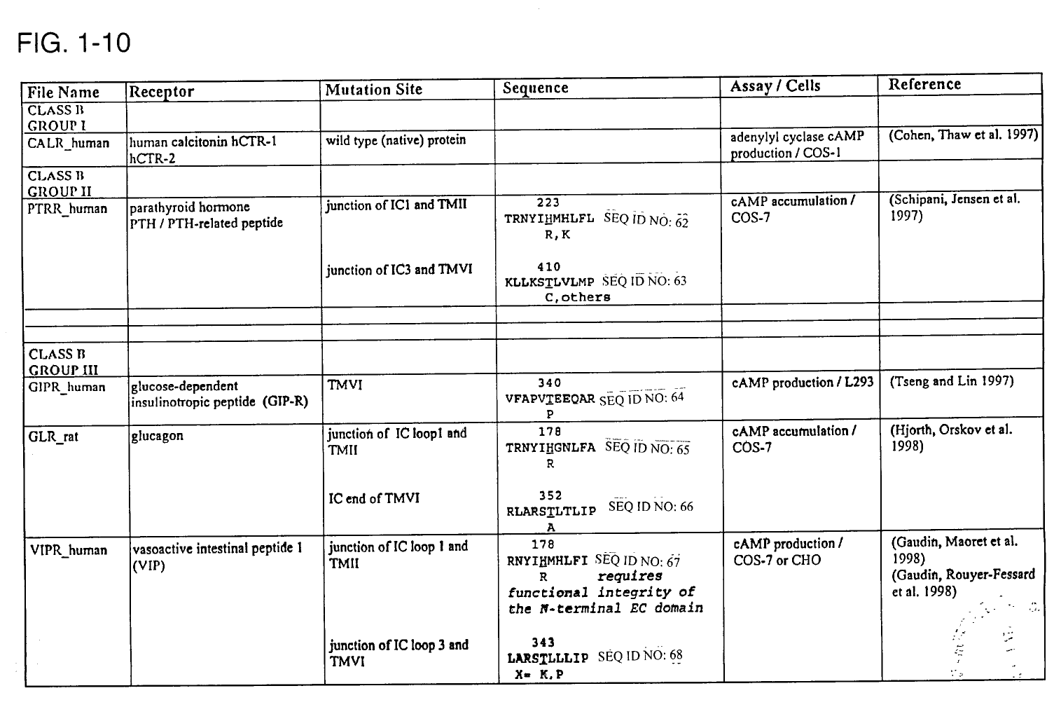

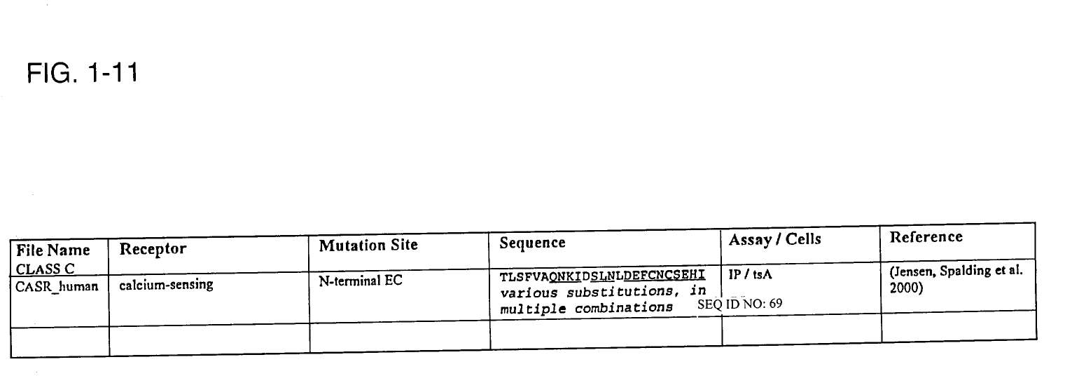

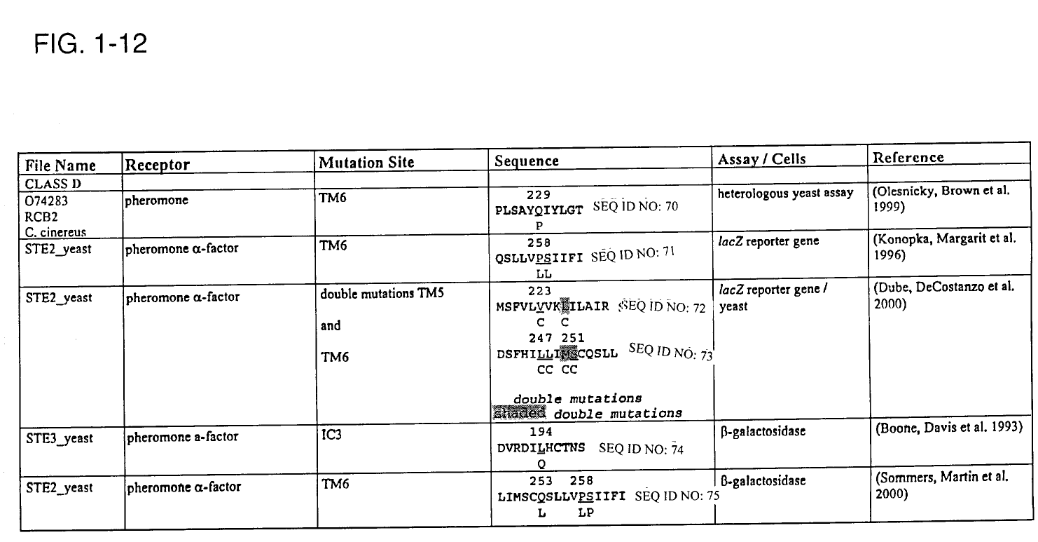

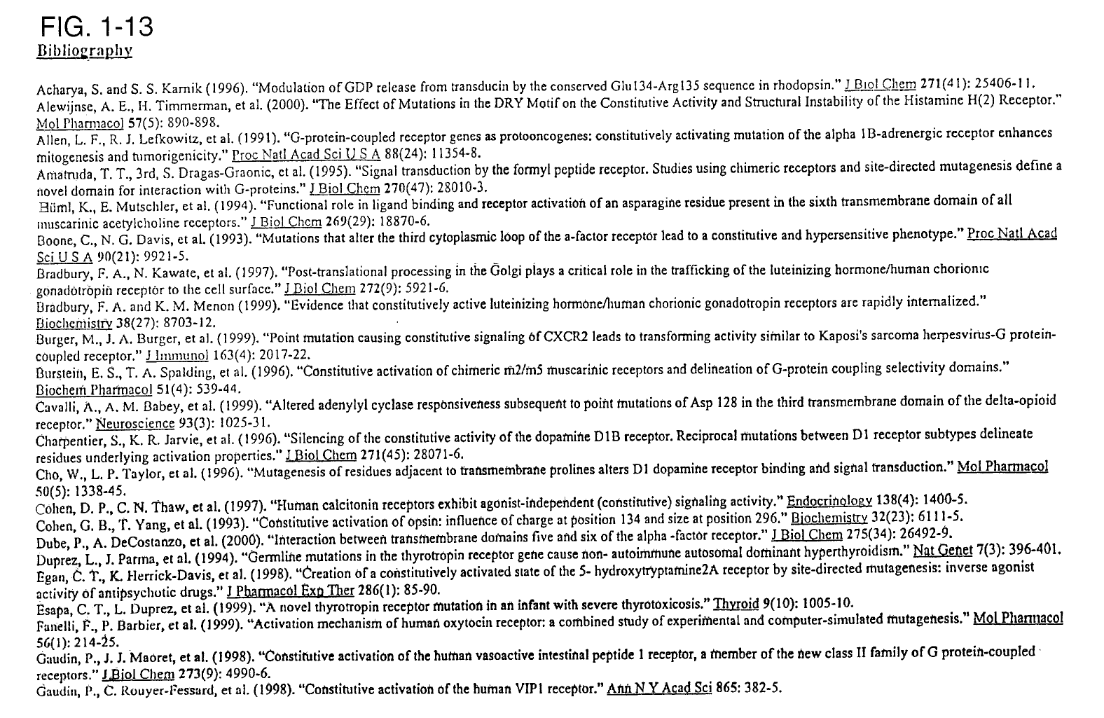

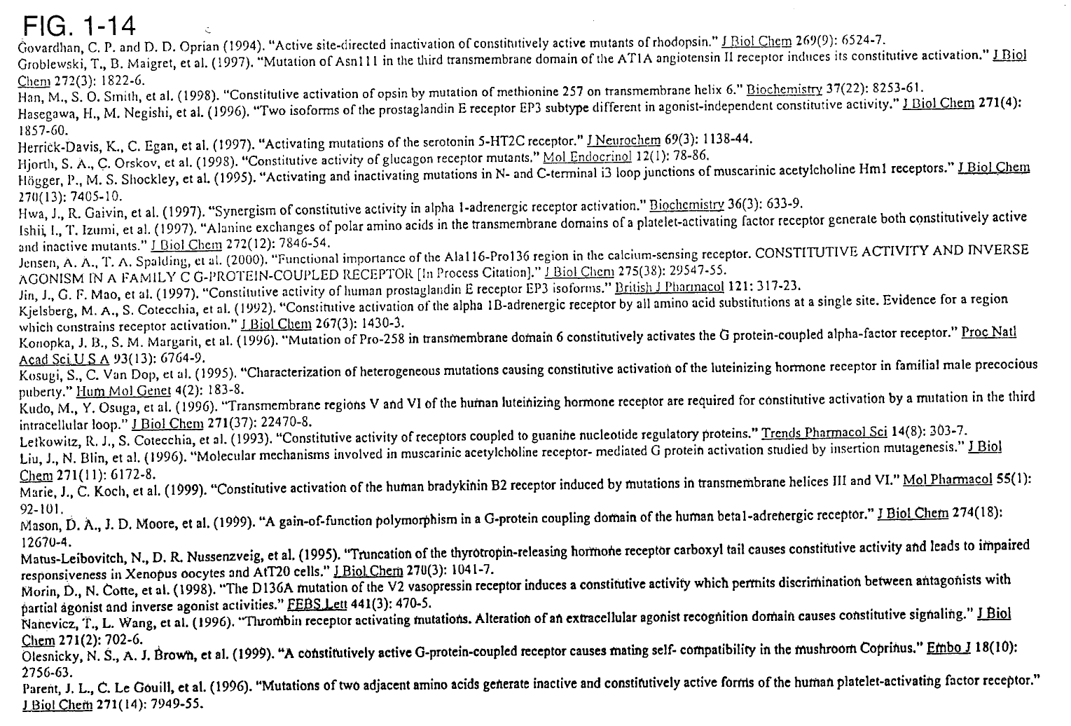

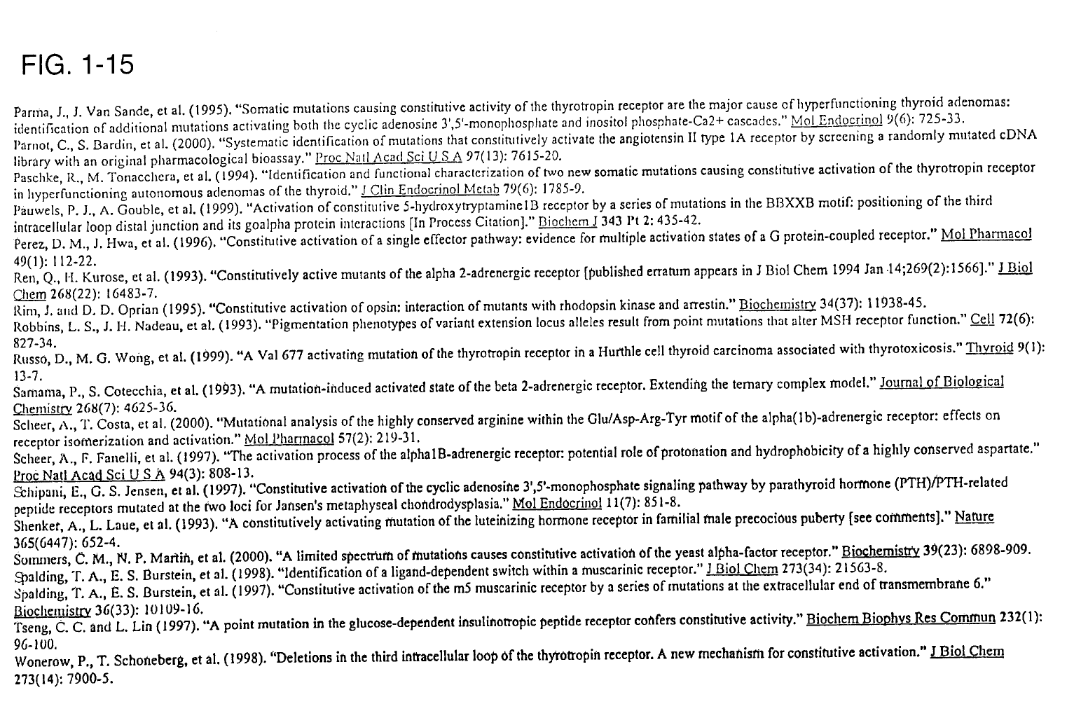

As will be appreciated by those skilled in the art, numerous constitutively active and hypersensitive receptors (naturally occurring and non-naturally occurring) have been previously identified. Such receptors provide a wealth of information that can be used to identify additional constitutively active, hypersensitive, or nonfunctional receptors. To complete step 1) above, available nucleic acid and/or amino acid sequence information, preferably amino acid sequence information, including wild-type and mutant receptors, is compiled to generate a database of constitutively active, hypersensitive, or nonfunctional receptor sequences. Next, the sequence of a given receptor (including any orphan receptor, non constitutively active receptor, non hypersensitive receptor, or functional receptor) of therapeutic interest (e.g., a receptor known to be a receptor for an agonist) is compared to the many sequences of constitutively active, hypersensitive, or nonfunctional receptors in the particular database to identify regions that are conserved between the receptor of therapeutic interest and the one or more constitutively active, hypersensitive, or nonfunctional receptors. The present invention demonstrates step 1) by providing an extensive database of constitutively active Class A G protein-coupled receptors (see FIG. 1). One of ordinary skill in the art will appreciate that additional databases may easily be generated for other types of receptor molecules, for example, Class B G protein-coupled receptors (see Jüppner et al., Curr. Opin. Nephrol. Hypertens. 3(4):371-378, Fig. 1, p 373 (1994)).

In order to complete step 2), for example, specific residues in a nonconstitutively active wild-type receptor are targeted for mutation based on the identified regions of homology between the nonconstitutively active receptor and constitutively active receptor(s), which are likely to impart constitutive activity onto the nonconstitutively active receptor. For example, if a region of homology between a nonconstitutively active receptor and a constitutively active receptor is identified that is identical in all amino acids but one, a mutation is introduced into the nonconstitutively active receptor to make the conserved region in the nonconstitutively active receptor identical to that of the constitutively active receptor. Alternatively, if the region conserved between the nonconstitutively active receptor and the constitutively active receptor shows a high degree of amino acid similarity, a series of targeted mutations are introduced into the nonconstitutively active receptor that are likely, based on the degree of homology and the knowledge of the skilled artisan, to make the receptor constitutively active. As but another example, the nonconstitutively active receptor might share a region of homology with another nonconstitutively active receptor that has been made constitutively active by the introduction of a certain mutation or mutations. In this case, the same or similar mutations are introduced into the given nonconstitutively active receptor.

Similarly, one skilled in the art will appreciate that in order to complete step 2) with a hypersensitive receptor, the same steps described above for a constitutively active receptor would be carried out for a hypersensitive receptor. For example, specific residues in a nonhypersensitive wild-type receptor are targeted for mutation based on the identified regions of homology between the nonhypersensitive receptor and hypersensitive receptor(s), which are likely to impart hypersensitivity onto the nonhypersensitive receptor. The candidate hypersensitive receptors are then stimulated with a low concentration of ligand (below saturating levels of ligand) and the receptor induced signal is measured. An increase in ligand-stimulated activity compared to the wild-type receptor indicates the identification of a hypersensitive receptor. A nonfunctional receptor may be similarly generated and tested for an absence or decrease in ligand-stimulated activity compared to the functional, wild-type receptor.

Alternatively, the database is used to identify regions of homology between a naturally occurring receptor of therapeutic interest and one or more constitutively active, hypersensitive, or nonfunctional receptors. The identified regions of homology would lead the skilled artisan to test the naturally occurring receptor for constitutive, hypersensitive, or non functional activity.

Applicants demonstrate step 2) by using the database of constitutively active Class A G protein-coupled receptors provided in step 1) (FIG. 1) to target specific residues in nonconstitutively active receptors for mutation. Briefly, highly conserved regions were identified between several nonconstitutively active receptors and a number of constitutively active Class A G protein-coupled receptors in the database. This information was used to target specific residues in the nonconstitutively active receptors for mutation. As described in detail below, targeted point mutations were introduced into the cholecystokinin-B/gastrin receptor (CCK-BR), the MC-4 receptor, and the mu opioid receptor which imparted constitutive activity to the nonconstitutively active receptors (see Examples 1, 2, and 3). It will be appreciated that this method of comparing nonconstitutively active receptors and constitutively active receptors to identify regions of conservation may be repeated with any family of related receptors with the goal of targeting regions of homology for mutation, as set forth in steps 1) and 2) above.

Step 3) involves assaying the mutant receptors for constitutive, hypersensitive, or nonfunctional activity by assaying for an increase in basal activity of the receptor. Of course, it will be appreciated that the constitutive activity, hypersensitivity, or lack of activity, respectively, of a particular receptor can be measured by any assay typically used to measure the basal and/or ligand-stimulated activity of the receptor. Any receptor of therapeutic interest will have such an associated assay, and such examples are provided herein (see Examples 1-10). To name but a few, changes in basal level second messenger signaling may be assessed to identify constitutively active receptors, including, but not limited to changes in basal levels of cAMP, cGMP, ppGpp, inositol phosphate, or calcium ion.

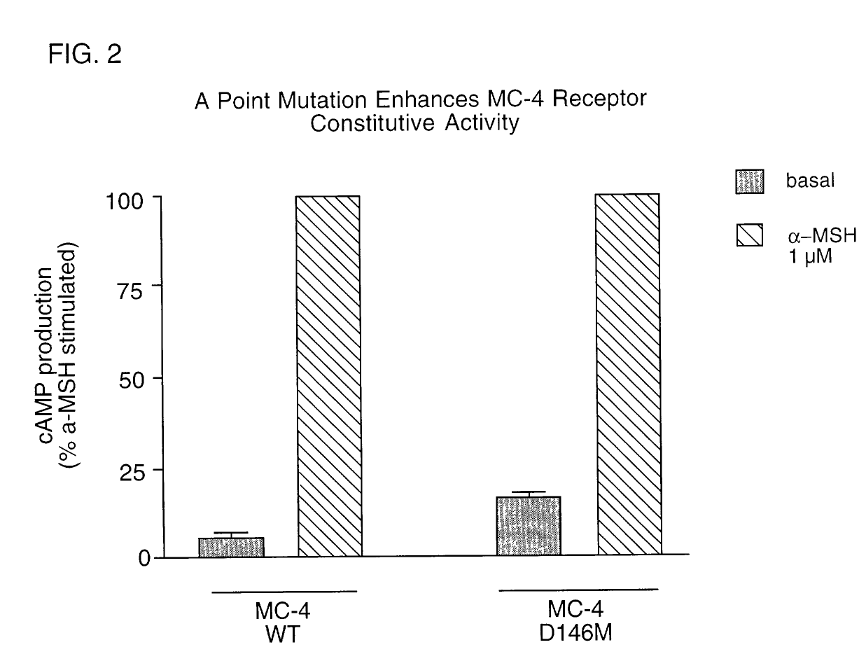

As but one example, ligand-dependent activation of the melanocortin-4 (MC-4) receptor is assayed by measuring an increase in cAMP production (Huszar et al., Cell 88:131-141, (1997)). The present invention demonstrates the use of this assay to identify a constitutively active MC-4 receptor (see FIG. 2). Specifically, the assay detected an increase in basal level cAMP production in a mutant MC-4 receptor; this mutant receptor was generated based on the homology of the wild-type MC-4 receptor to other constitutively active Class A G protein-coupled receptors.

These simple principles can easily be applied to identify additional constitutively active G protein-coupled receptors. For example, similar studies that measured increases in intracellular cAMP were carried out to identify constitutively active mutants of the pituitary adenylate cyclase activating polypeptide type I receptor (PAC1) (Cao et al., FEBS Lett., Mar. 10;469(2-3):142-146, (2000)). As but another example, the constitutively active mutants of the β2 bradykinin (BK) receptor and the AT1A angiotensin I and II receptors were identified by measuring inositol phosphate production (Marie et al., Mol. Pharmacol. 1:92-101, (1999); Groblewski et al., J. Biol. Chem., 272(3):1822-1826, (1997); Feng et al., Biochemistry, 37(45):15791-15798 (1998)). A constitutively active CCK-BR was also identified by measuring basal inositol phosphate production (Beinborn et al., J. Biol. Chem. 273(23): 14146-14151 (1998); and Fig. 1). Mutants of CCK-BR were tested by simply comparing the basal level of inositol phosphate production of a mutant CCK-BR to the basal level inositol phosphate production of the wild-type CCK-BR to determine whether the mutant CCK-BR was constitutively active.

Additional examples of G protein-coupled receptors having intracellular second messenger signaling pathways that may be evaluated to identify constitutively active forms of receptors include the GLP-1 receptor (adenylate cyclase and phospholipase C (PLC)) and the parathyroid hormone receptor (PTH) (see Dillon et al., Endocrinology 133(4):1907-1910, (1993); Whitfield and Morley, TiPS, 16:382-385, 1995). Other G protein-coupled receptors bind to certain intracellular molecules in their activated states. For example, the mu opioid receptor induces an increased level of GTP binding by receptor-activated G protein (Gαi) (see, e.g., Befort et al., J. Biol. Chem. 274(26):18574-18581, (1999)).

The activity of other types of receptors (e.g., non-G protein-coupled receptors such as single transmembrane domain receptors and nuclear receptors) can also be measured via the biochemical pathway they induce. For example, binding of the ligand EPO to the EPO receptor activates the JAK2-STAT5 signaling pathway (see, e.g., Yoshimura et al., Curr. Opin. Hematol., 5(3):171-176, 1998). The basal and stimulated levels of JAK2 and STAT5 signaling can easily be assessed by one of ordinary skill in the art, as described in Yoshimura et al., supra, to identify constitutively active (or hypersensitive) EPO receptors.

As an alternative to measuring molecules in a signaling pathway directly to identify constitutively active, hypersensitive, and nonfunctional receptors, a reporter assay system may be established in which a response element, responsive to signaling through a particular receptor, is attached to a reporter gene in combination with a transcriptional promoter. Specifically, the expression of the reporter gene is controlled by the activity of the chosen receptor. This method involves the steps of 1) identifying a response element that is sensitive to signaling by a specific receptor polypeptide (e.g., by eliciting an increase or decrease in gene expression upon receptor activation); 2) operably linking the response element and a promoter to a reporter gene; and 3) comparing the basal or ligand-stimulated reporter activity of a candidate receptor to a negative control. An increase in the basal level reporter activity compared to the negative control indicates the identification of a constitutively active receptor. Similarly, an increase in ligand stimulated activity, compared to the negative control, indicates the identification of a hypersensitive receptor, and an absence of ligand-stimulated activity, compared to a corresponding functional receptor, indicates the identification of a nonfunctional receptor. It is important to note that hypersensitive receptors may not necessarily have any detectable increase in basal activity. In preferred embodiments, this assay system is used to screen for receptor mutants exhibiting constitutive, hypersensitive, or nonfunctional activity.

It will be appreciated that the receptor can be any receptor identified as a candidate constitutively active, hypersensitive, or nonfunctional receptor. In addition, one skilled in the art will recognize that the response element used in the present response assay can be any response element that is sensitive to signaling through the identified candidate receptor. For example, in reporter assays for identifying constitutively active receptors that are coupled to different G proteins, one would select response elements that are sensitive to signaling downstream of respective G proteins. Examples of preferred response elements include a portion of the somatostatin promoter (which has included a number of different response elements) (SMS), the serum response element (SRE), and the cAMP response element (CRE), which are response elements sensitive to G protein-coupled receptor signaling. Other preferred response elements include response elements sensitive to signaling through a single transmembrane receptor or a nuclear receptor. In particular examples, SMS is activated by coupling of receptors to either Gαq or Gαs; SRE is activated by receptor coupling to Gαq; and CRE is activated by receptor coupling to Gαs and inhibited by coupling to Gαi; and the TPA response element (sensitive to phorbol esters) is activated by receptor coupling to Gαq. Each of these response elements can be employed in a reporter assay to generate a readout for the basal and ligand-stimulated activity of a specific G protein-coupled receptor.

More generally, a reporter construct for detecting receptor signaling might include a response element that is a promoter sensitive to signaling through a particular receptor. For example, the promoters of genes encoding epidermal growth factor, gastrin, or fos can be operably linked to a reporter gene for detection of G protein-coupled receptor signaling. Another example includes the TPA response element, which is sensitive to phorbol ester induction.

It will be appreciated that a wide variety of reporter constructs can be generated that are sensitive to any of a variety of signaling pathways induced by signaling through a particular receptor (e.g., a second messenger signaling pathway). Accordingly, this assay system may be used to identify other types of constitutively active, hypersensitive, or nonfunctional receptors, including receptors that are single transmembrane receptors or nuclear receptors, by simply selecting a response element that is sensitive to the particular receptor and positioning the response element upstream of a reporter gene in a reporter construct. For example, the elements AP-1, NF-κb, SRF, MAP kinase, p53, c-jun, TARE can all be positioned upstream of a reporter gene to obtain reporter gene expression. Additional response elements, including promoter elements, can be found in the Stratagene catalog (PathDetect® in Vivo Signal Transduction Pathway cis-Reporting Systems Introduction Manual or PathDetect® in Vivo Signal Transduction Pathway trans-Reporting Systems Introduction Manual, Stratagene, La Jolla, Calif.).

In preferred embodiments, the G protein-coupled reporter assay system includes 1) a reporter construct containing a response element that is sensitive to signaling through a specific G protein, and a promoter, operably linked to a reporter gene; preferably in combination with 2) an expression vector containing a promoter operably linked to a nucleic acid encoding a receptor, wherein the receptor is coupled to a G protein or other downstream mediator to which the selected response element is sensitive. Alternatively, a G protein-coupled receptor assay includes transfection of wild-type or polymorphic receptors into cells followed by assessment of the levels of transcription of cell specific genes compared to the appropriate controls (e.g., transfected cells compared to nontransfected cells and the presence or absence of ligand stimulation).

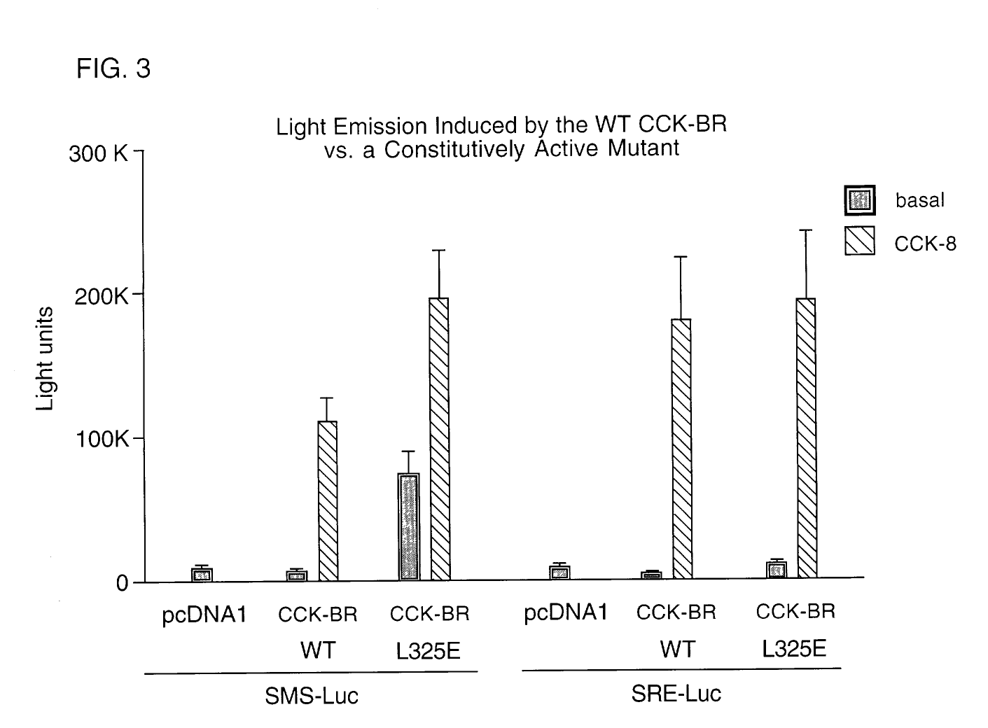

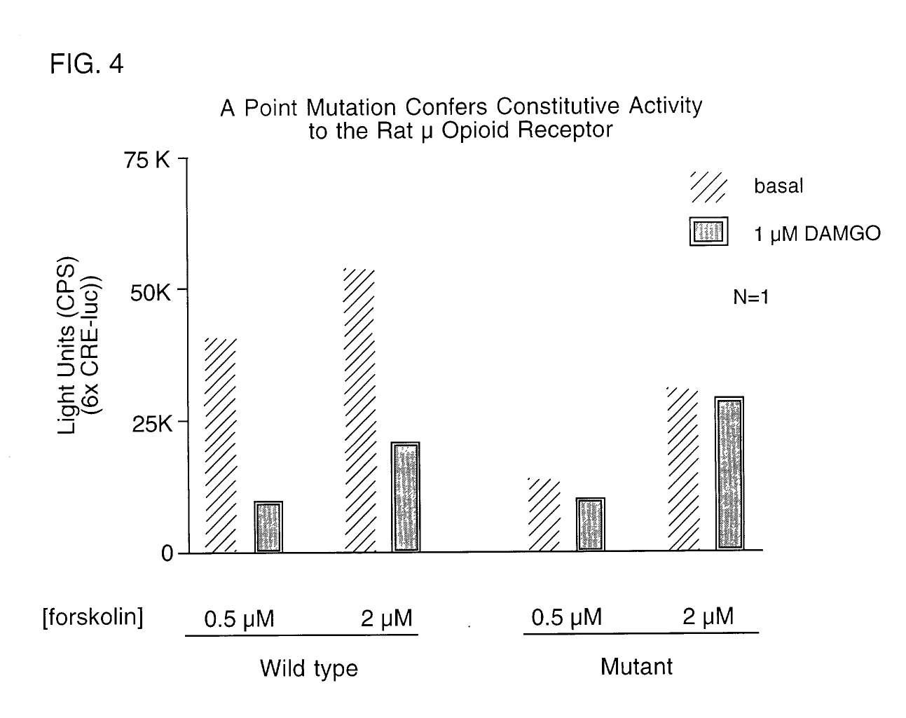

The experiments described herein demonstrate the use of specific response elements that are sensitive to signaling through each of Gαq, Gαs, and Gαi. For example, the SMS and SRE response elements each detect an increase in basal activity of the Leu325Glu CCK-BR mutant receptor, which is coupled to Gαq (see FIG. 3). Similarly, a constitutively active rat mu opioid receptor was identified using a reporter construct sensitive to Gαi coupling (see FIG. 4). The response element employed in this assay was the cAMP-response element (CRE), which is sensitive to Gαi mediated reductions in intracellular levels of cAMP. Signaling through the rat mu opioid receptor via Gαi inhibits adenylate cyclase, causing a decrease in intracellular cellular cAMP. Therefore, an increase in rat mu opioid receptor signaling induces a decrease in CRE mediated reporter activity.

Mutation induced Gαi-mediated decreases in intracellular cAMP were, prior to the present invention, more often measured by 1) stimulating cells with forskolin, which causes receptor-independent activation of adenylate cyclase and generates an intracellular pool of cAMP; 2) stimulating the cells with ligand; and 3) measuring the ligand-induced, receptor-dependent Gαi-mediated decrease in the intracellular cAMP pool (e.g., using a radioimmunoassay (e.g., New England Nuclear, Boston, Mass.)). As demonstrated herein, the reporter system approach was capable of identifying a constitutively active rat mu opioid receptor (FIG. 4). Specifically, cells transfected with a CRE-Luc reporter construct (Stratagene, La Jolla, Calif.) and an expression vector encoding either a wild-type or a mutant rat mu opioid receptor were stimulated with 0.5 μM or 2 μM forskolin to increase the intracellular pool of cAMP. The basal (and ligand-induced) level of receptor activity was then measured using a standard luciferase assay (see FIG. 4). Coexpression of the receptor of interest with a luciferase reporter gene construct allows one to measure light emission as a readout for basal signaling.

The results illustrated in FIG. 4 show a reduction in basal activity (i.e., forskolin-induced cAMP production in the absence of receptor stimulation) when the expressed mutant rat mu opioid receptor is compared to the basal activity of the expressed wild-type rat mu opioid receptor. This decrease in activity indicates an increase in the basal level activity of the mutant rat mu opioid receptor, because activation of the rat mu opioid receptor induces a decrease in CRE-mediated reporter activity (FIG. 4, compare 0.5 μM wild-type vs. 0.5 μM mutant and 2 μM wild-type vs. 2 μM mutant).

It is important to note that the level of constitutive activity in the mutant rat mu opioid receptor is increased to 50% of the level of ligand-stimulated activity of the wild-type receptor. This high level of inhibitory signaling supports the hypothesis that constitutively active receptors, introduced by gene therapy, are likely to transduce a sufficient intracellular signal to reduce pain in vivo. According to the present invention, even low levels of basal signaling may mimic the effect of the ligand-stimulated signaling achieved with endogenous concentrations of agonist. For example, the signal transduced in a cell in vivo is likely to be less than the ligand-stimulated signal measured experimentally. This may be due to the low in vivo concentrations of endogenous ligand or to the low in vivo levels of expression of the receptor on the surfaces of cells. It will be appreciated that these features can be manipulated to control the level of constitutive activity transduced by the cell. For example, for a weak constitutively active receptor, the level of expression can be increased to achieve increased signaling, for example, by selecting a strong constitutive promoter. Alternatively, for a strong constitutive receptor, a high level of expression might not be required to achieve sufficient signaling. Alternatively, signaling might be diminished by reducing the level of expression of the strong constitutively active receptor.

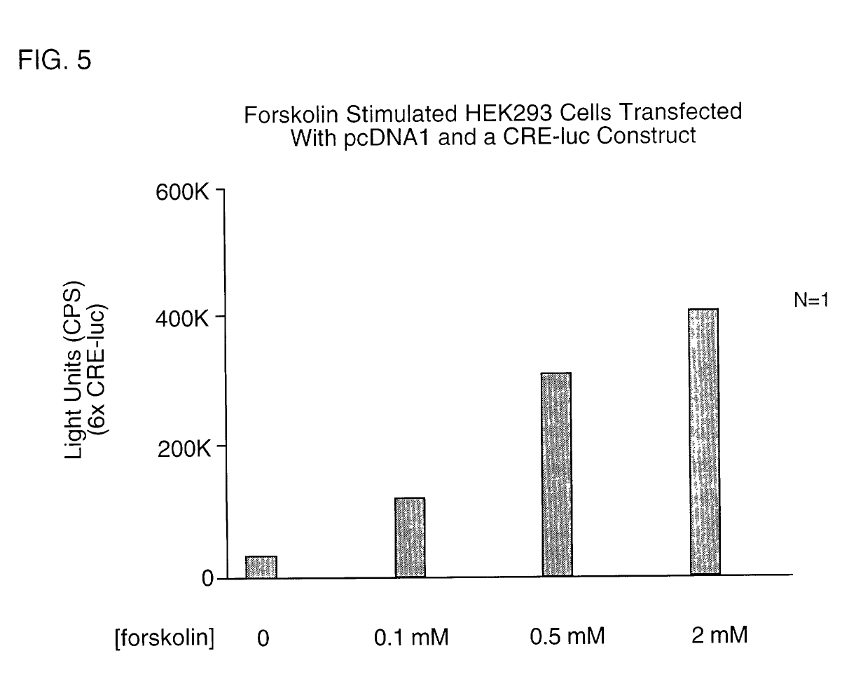

Although successful, use of the prior method of measuring Gαi coupling has several disadvantages. First, detecting Gαi mediated inhibition of cAMP requires overcoming the simultaneous positive effects of forskolin on adenylate cyclase. For example, FIG. 5 illustrates the positive effect of forskolin in HEK293 cells on the response of CRE-Luc in the absence of a contransfected receptor protein. In addition, detection of a ligand-stimulated decrease in intracellular cAMP relies on whether a large enough percentage of the cells are successfully transfected with, and express, the receptor molecule. Moreover, when using transient transfection assays, interexperimental variation occurs because the percentage of cells transfected from one experiment to the next is difficult to control.

A positive assay for Gαi coupling (i.e., one that yields an increase in luciferase activity upon receptor activation, instead of a negative assay, one that yields a decrease in luciferase activity upon receptor activation), provides a detectable output signal and less interassay variation. It was hypothesized that Gαi coupling could be detected by altering the signaling pathway generated by Gαi coupled receptors. A chimeric G protein (Gqi5, Broach and Thorner, Nature 384 (Suppl.): 14-16 (1996)) that contains the entire Gαq protein having five C-terminal amino acids from Gαi attached to the C-terminus of Gαq has been generated. This chimeric G protein is recognized as Gαi by Gαi coupled receptors, but switches the receptor induced signaling from Gαi to Gαq. This allows Gαi receptor coupling to be detected using a positive assay by use of the Gαq responsive SMS-Luc or SRE-Luc construct (Stratagene, La Jolla, Calif.). SMS and SRE preferably respond to Gαq mediated inositol and calcium production. Moreover, detection can be carried out in the absence of forskolin pre-stimulation of cells.

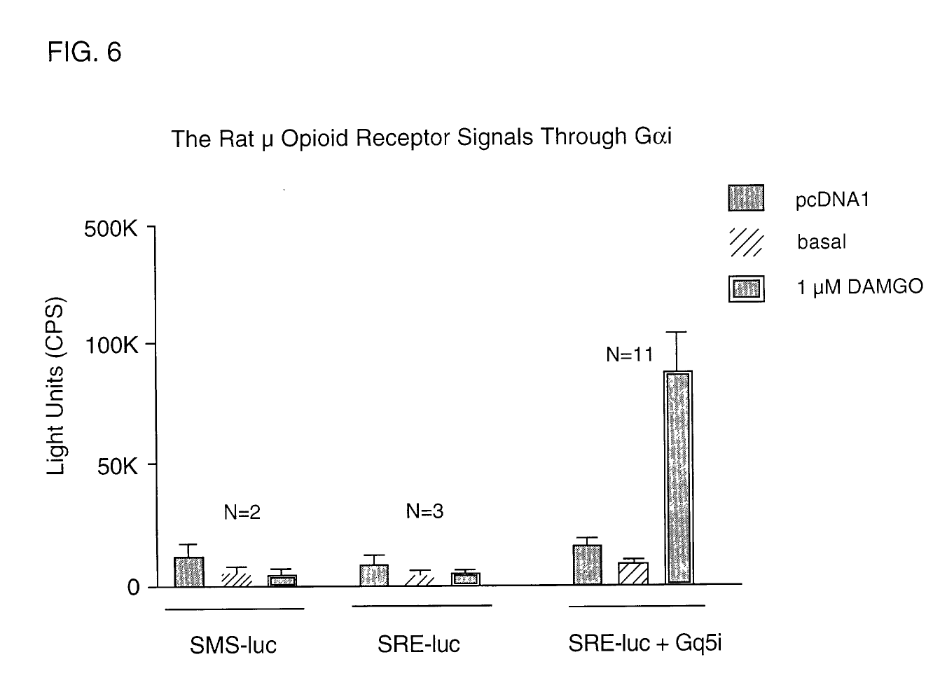

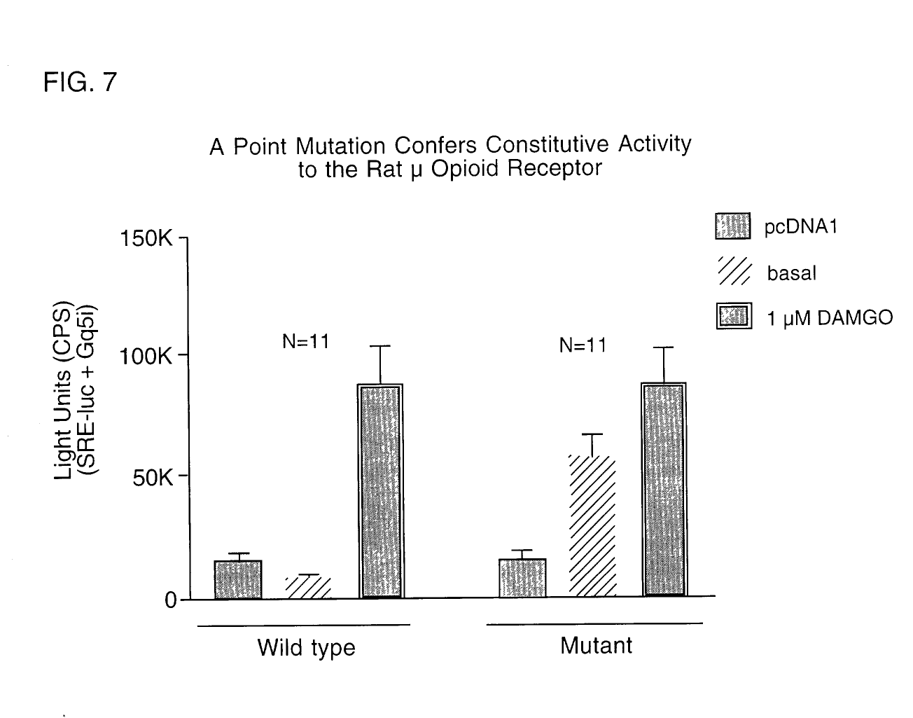

As demonstrated in FIG. 6, Gq5i can be used to detect rat mu opioid receptor coupling to Gαi . FIG. 6 shows that no ligand-stimulated luciferase activity is detected in response to ligand stimulation using luciferase constructs having either the SMS or SRE alone (left two columns), whereas a large increase in ligand-stimulated luciferase activity is detected using SRE-Luc in combination with Gq5i (far right). This assay was also employed to measure the constitutive activity of the Asn150Ala mutant rat mu opioid receptor (FIG. 7).

One skilled in the art will appreciate that the assays described herein for the various constitutively active receptors can also be applied in the identification of hypersensitive or nonfunctional receptors. More particularly, any assay that measures the ligand-stimulated response of a particular receptor can be used to identify hypersensitive or nonfunctional receptors. For example, a hypersensitive receptor may be identified by exhibiting a ligand-dependent increase in intracellular signaling compared to the wild-type receptor. More specifically, a hypersensitive receptor may be characterized in that it exhibits an increased response to a specific concentration of ligand, compared to the response of a wild-type receptor to the same concentration of ligand. For example if 5 μM ligand induces a 5-fold stimulation of activity in a wild-type receptor, compared to a negative control, 5 μM ligand may stimulate a 10-fold stimulation in activity in a hypersensitive receptor, compared to the same negative control. As noted above, a hypersensitive EPO receptor has been identified using such assays (Watowich et al. supra).

Furthermore, a number of examples are provided herein that illustrate the ease with which these and similar approaches can be applied to identify non-G protein-coupled constitutively active, hypersensitive, or nonfunctional receptors, including, constitutively active, hypersensitive, or nonfunctional single transmembrane domain receptors (e.g., EPOR, see Example 11) and nuclear receptors (steroid hormone receptors, see Example 10).

Mu Opioid Receptor

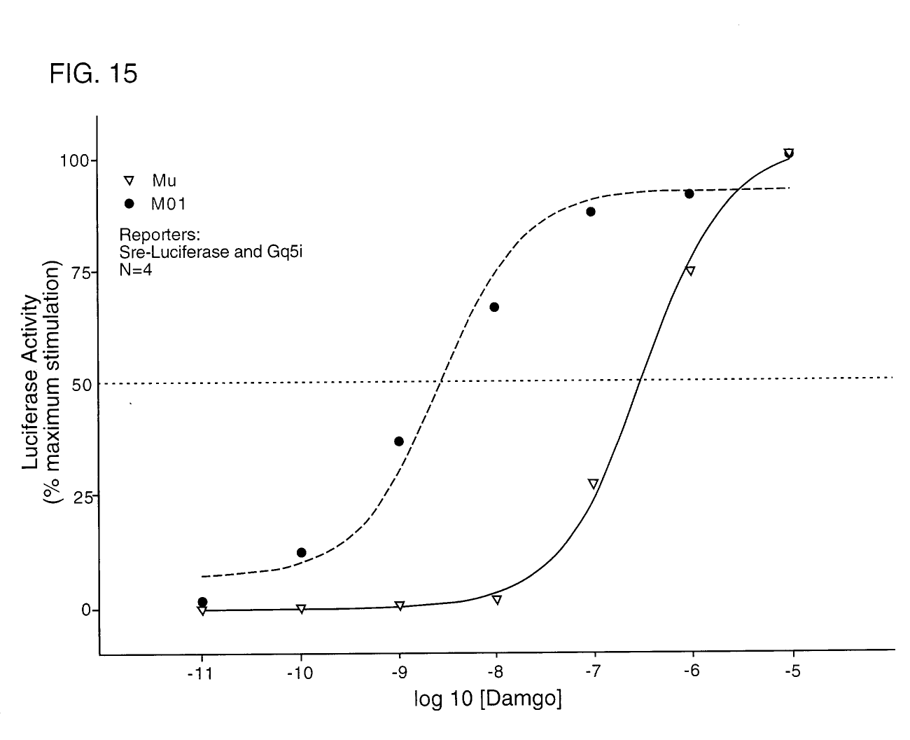

According to one preferred embodiment of the present invention, nucleic acids are identified that encode clinically useful constitutively active receptors. We demonstrate this aspect of the invention by identifying a constitutively active mu opioid receptor. It is important to note that the mu opioid receptor of the invention is also hypersensitive. For example, the affinity of the mu opioid receptor for the ligand DAMGO is increased (see FIG. 15, which shows that a mutation that confers constitutive activity to the mu opioid receptor also confers hypersensitivity; the mutant receptor is responsive to a lower concentration of ligand than the wild-type receptor).

The mu opioid receptor is an opiate receptor that falls within the G protein-linked seven transmembrane domain neuropeptide receptor family. In general, opiate receptors (including μ (mu), κ, δ, and opiate-like receptor (OLR)) couple to guanine nucleotide binding (G) proteins (Li et al. supra). For example, opiates can alter GTP hydrolysis, GTP analogs and pertussis toxin can change opiate receptor binding, and opiates can influence G-protein-linked second messenger systems and ion channels. More specifically, mu opioid receptors have a characteristic high affinity for morphine and other opiate drugs and peptides. Binding of morphine to the mu opioid receptor results in an analgesic and euphoric effect, common to opiate drugs. In the present invention, the mu opioid receptor is of particular interest because of its analgesic properties. The present invention provides a method of administering a nucleic acid encoding a constitutively active morphine receptor to a patient in pain to provide significant relief from the pain, while reducing the side effects experienced upon administration of morphine.

A single point mutation (Asn to Ala at amino acid 150) was introduced into the third transmembrane region of the rat mu opioid receptor (SEQ ID NO: 1). This Asn residue was targeted for mutation based on it being highly conserved between the mu opioid receptor, the bradykinin B2 receptor, and the angiotensin II AT1A receptor. Furthermore, homologous mutations at this residue in the bradykinin B2 and angiotensin II AT1A receptors yielded receptors having constitutive activity. Indeed, the Asn150Ala mu opioid receptor mutant exhibited levels of basal activity, which exceeded 50% of the maximal level of ligand-stimulated second messenger signaling (see Example 1).

According to the present invention, the constitutively active mu opioid receptor described herein may be inserted into any of a variety of known viral and non-viral vectors and administered to a particular cell, tissue, or site in a mammal to obtain therapeutic benefit. A particularly preferred viral vector is the adenoviral vector. In fact, the adenoviral vector has been used previously for gene therapy to reduce pain and to introduce genes of interest into the intrathecal space (the fluid that bathes the spinal cord) (see Burcin et al., Proc. Natl. Acad. Sci. USA 96:355-360, (1999); Finegold et al., Human Gene Therapy 10:1251-1257, (1999); Vasquez et al., Hypertension October Part II 756-761 (1999); Mannes et al., Brain Research 793:1-6, (1998)).

In preferred embodiments, expression of the constitutively active mu receptor results in an analgesic response at the site of administration. In one particularly preferred embodiment, a virally encoded constitutively active mu opioid receptor is used to treat patients with chronic back pain resulting from any etiology, including fracture or metastatic disease. Alternatively, the pain is due to arthritis or other inflammatory diseases. For example, an adenoviral construct encoding the constitutively active mu opioid receptor may be administered into the intrathecal space for treatment of pain, for example, back pain. It will be appreciated that a nucleic acid encoding a constitutively active mu opioid receptor may be delivered to a patient experiencing pain in any location in the body.

Also included is the administration of constitutively active, hypersensitive, or nonfunctional allelic variations, natural mutants, or induced mutants of mu opioid receptors. Of particular interest are mu opioid receptor mutants in which the mutation is at or near the region surrounding the N residue at position 150 of SEQ ID NO: 1, at or surrounding the DRY motif, at positions 154-156 of SEQ ID NO:1, or at or surrounding positions 13 and 20 residues N-terminal to the CWLP motif of SEQ ID NO: 1. The invention also includes the use of nucleic acids encoding chimeric polypeptides that contain, as part of the chimera, the mu opioid receptor polypeptide (e.g., in addition to G protein).

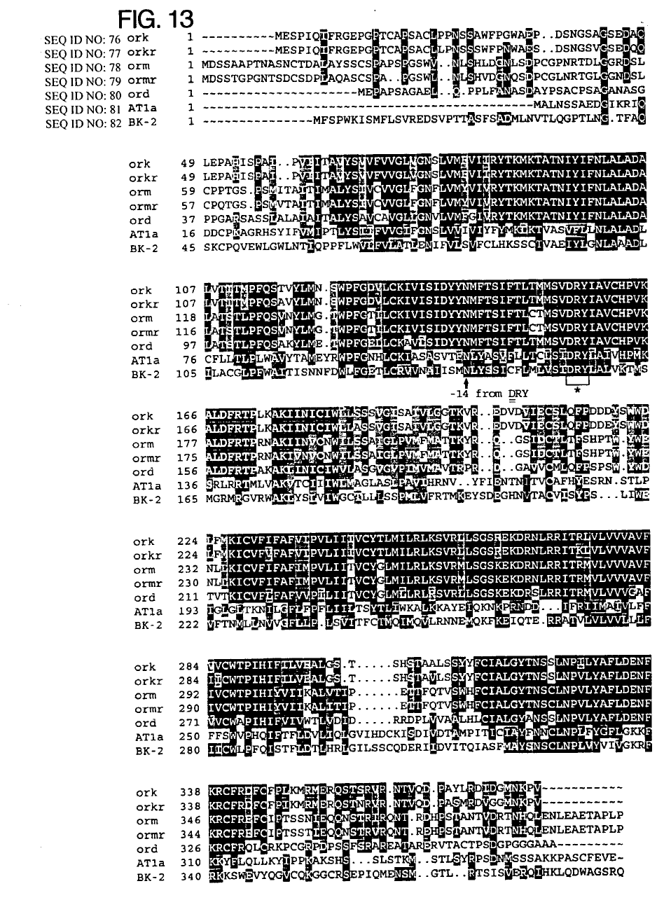

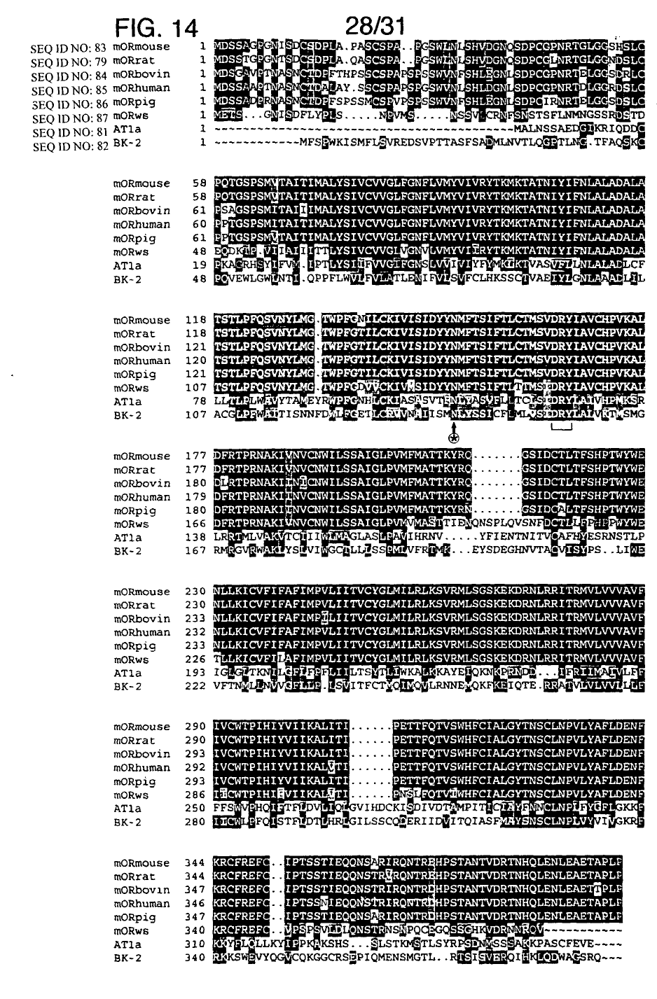

The invention further includes nucleic acids encoding any constitutively active or hypersensitive fragment or analog of the mu opioid receptor, or any other constitutively active receptor identified by methods described herein. A constitutively active fragment or analog of the mu opioid receptor possesses in vivo or in vitro basal activity, which is greater than the wild-type basal activity (see in FIGS. 13, 14 and 7, and SEQ ID NO: 1). A useful constitutively active mu opioid receptor fragment or constitutively active mu opioid receptor analog is one that exhibits constitutive biological activity in any biological assay for mu opioid receptor activity (for example, those assays described in Example 1).

It will be appreciated that nucleic acids encoding any constitutively active, hypersensitive, or nonfunctional receptor, e.g., any Class A G protein-coupled receptor (e.g., MC-4 or CCK-BR) or Class B G protein-coupled receptor (GLP-1 or PTH), any single transmembrane domain receptor (e.g., EPOR), or any nuclear receptor (e.g., steroid hormone receptors, such as the estrogen receptor), can also be utilized as gene therapeutic agents, and such is within the ability of one skilled in the art.

Viral Vectors for Gene Delivery

Viral vectors are primary gene transfer tools for gene therapy and other gene transfer applications using both ex vivo and in vivo protocols. Viral vectors, particularly retroviral vectors with the appropriate tropisms for the selected cells are particularly useful for therapeutic delivery of nucleic acids and may be used as gene transfer delivery systems for the constitutively active, hypersensitive, or nonfunctional receptors of the present invention. Numerous vectors useful for this purpose are generally known and have been described (Miller, Human Gene Therapy 15:14 (1990); Friedman, Science 244:1275-1281 (1989); Eglitis and Anderson, BioTechniques 6:608-614 (1988); Tolstoshev and Anderson, Current Opinion in Biotechnology 1:55-61 (1990); Sharp, The Lancet 337:1277-1278 (1991); Cornetta et al., Nucleic Acid Research and Molecular Biology 36:311-322 (1987); Anderson, Science 226:401-409 (1984); Moen, Blood Cells 17:407-416 (1991); and Miller and Rosman, Biotechniques 7:980-990(1989)) incorporated by reference herein. Retroviral vectors are particularly well developed and have been used in the clinical setting to provide therapeutic benefit (Rosenberg et al., N. Engl. J. Med 323:370 (1990)).

Viral vectors of the present invention include viral nucleic acids (e.g., DNA or RNA) that have been modified to serve as vectors for nucleic acids encoding constitutively active, hypersensitive, or nonfunctional receptors. Viral vectors of the present invention include any viral vector having the ability to transfer (or “transduce”) a nucleic acid to a cell by infecting that cell. Viral vectors which may be utilized in the present invention include adenoviral vectors and adeno-associated virus-derived vectors (Burcin et al., supra; Finegold et al., supra; Vasquez et al. supra; Mannes et al. supra; Ilan et al., Seminars in Liver Disease, 19:49-59, (1999); Patijn et al., Seminars in Liver Disease 19:61-39, 1999), retroviral vectors (e.g., Moloney Murine Leukemia virus based vectors, Spleen Necrosis Virus based vectors, Friend Murine Leukemia based vectors (Ganjam, Seminars in Liver Disease, 19:27-37 (1999)), lentiviral based vectors (Human Immunodeficiency Virus based vectors etc.), papova virus based vectors (e.g., SV40 viral vectors, see e.g., Strayer et al., Seminars in Liver Disease, 19:71-81 (1999), Herpes-Virus based vectors, viral vectors that contain or display the Vesicular Stomatitis Virus G-glycoprotein Spike, Semi-Forest virus based vectors, Hepadnavirus based vectors, and Baculovirus based vectors. Particularly preferred viral vectors include adenoviral vectors. Moreover, the technique of the present invention is not limited to gene-delivery vectors, but also to whole, naturally occurring viruses upon which the above-mentioned vectors are based. The adenoviral vector delivery system for nucleic acids encoding the mu opioid or other constitutively active, hypersensitive, or nonfunctional receptors is particularly useful because the adenovirus has been shown to be easily distributed to a particular site upon direct injection to that site (including neuronal sites like the intrathecal space, see Finegold et al., supra and Mannes et al. supra).

The retroviral constructs, packaging cell lines, and delivery systems which may be useful for this purpose include, but are not limited to, one, or a combination of the following: self inactivating vectors; double copy vectors; selection marker vectors; and suicide mechanism vectors.

Fragments or analogs of the constitutively active mu opioid or other receptors of the invention, may also be administered by any suitable viral vector system. Useful fragments or analogs of the mu opioid or other receptor may be administered by inserting the nucleic acid encoding the fragment or analog in place of the full length receptor gene into a gene therapy vector.

In preferred embodiments, a standard ex vivo viral gene therapy procedure may be useful in treating a mammal diagnosed with a disease. In ex vivo gene therapy, a specific cell type or tissue is removed from a subject and genetically engineered in vitro using viral gene transfer vectors. The genetically engineered cell or tissue is subsequently returned to the subject. In this type of gene therapy protocol, highly infectious viral vectors with broad tropisms, such as those with amphotropic envelope glycoprotein are particularly useful, (e.g., glycoprotein of the Moloney murine leukemia virus or glycoprotein G of the vesicular stomatitis virus (VSVG)). For example, in one preferred embodiment, a constitutively active, hypersensitive, or nonfunctional receptor of the present invention is administered to a subject using ex vivo gene therapy by (i) transfecting a selected cell type in vitro with nucleic acid encoding the selected receptor; (ii) allowing the cells to express the receptor; and (iii) administering the modified cells to an individual to generate a therapeutic effect in the individual.

Retroviral delivery of constitutively active, hypersensitive, or nonfunctional receptors, or other forms of gene transfer are also particularly appropriate for treatment of cancer (e.g., a constitutively active somatostatin receptor, or a nonfunctional growth factor receptor, to reduce growth of cancer cells), neoplasms of the immune system, as removal, treatment, and re-implantation of hematopoietic cells is a matter of course for the treatment of these neoplasms. Standard techniques for the delivery of gene therapy vectors may be used to transfect stem cells. Such transfection may result in cells that synthesize a constitutively active, hypersensitive, or nonfunctional receptor useful in lowering the recurrence rate of the neoplasm in the patient.

Non-Viral Gene Delivery

A wide variety of non-viral nucleic acid delivery techniques that can be used in vitro, in vivo, or ex vivo are also well known in the art. Nucleic acids encoding constitutively active, hypersensitive, or nonfunctional receptors, e.g., the mu opioid receptor, or a fragment or analog thereof, under the regulation of the appropriate promoter, and including the appropriate sequences required for insertion into genomic DNA of the patient, or autonomous replication, may be administered to the patient using the following gene transfer techniques: microinjection (Wolff et al., Science 247:1465 (1990)); calcium phosphate transfer (Graham and Van der Eb, Virology 52:456 (1973)); Wigler et al., Cell 14:725 (1978)); Felgner et al., Proc. Natl. Acad. Sci. USA 84:7413 (1987)); lipofection (Felger et al., supra; Ono et al., Neuroscience Lett. 117:259 (1990)); Brigham et al., Am. J. Med. Sci. 298:278 (1989)); Staubinger and Papahadjopoulos, Meth. Enz. 101:512 (1983)) asialorosonucoid-polylysine conjugation (Wu and Wu, J. Biol. Chem. 263:14621 (1998)); Wu et al., J. Biol. Chem. 264:16985 (1989)); electroporation (Neumnn et al., EMBO J. 7:841 (1980)); and receptor mediated endocytosis of DNA (Smith et al., Seminars in Liver Disease 19:83-92 (1999)). These references are hereby incorporated by reference.

For example, the nucleic acids encoding the constitutively active, hypersensitive, or nonfunctional receptors of the present invention may be associated with liposomes, e.g., such as lecithin liposomes or other liposomes known in the art, e.g., nucleic acid liposomes (for example, as described in WO 93/24640, incorporated herein by reference). Liposomes that include cationic lipids interact spontaneously and rapidly with polyanions, such as DNA and RNA, resulting in liposome/nucleic acid complexes. In addition, the polycationic complexes fuse with cell membranes, resulting in an intracellular delivery of polynucleotides that bypasses the degradative enzymes of the lysosomal compartment. This may be of particular use for administering RNA molecules. Published PCT application WO 94/27435, incorporated herein by reference, describes compositions for genetic immunization that include cationic lipids and polynucleotides. Agents that assist in the cellular uptake of nucleic acid, such as calcium ions, viral proteins, and other transfection facilitating agents, may advantageously be used.

Therapeutic Compositions

The present invention further provides compositions that include nucleic acids encoding constitutively active, hypersensitive, or nonfunctional receptors in a pharmaceutically acceptable carrier. Pharmaceutically acceptable carriers for use with the invention include aqueous solutions, non-toxic excipients, including water saline, dextrose, glycerol ethanol, buffers, and the like, (and combinations thereof) as described in Remington's Pharmaceutical Sciences, 15^{th} Ed. Easton: Mack Publishing Co. pp. 1405-1412 and 1461-1487 (1975) and The National Formulary XI., 14^{th} Ed. Washington: American Pharmaceutical Association (1975), the contents of which are incorporated herein by reference. Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobials, anti-oxidants, chelating agents, and inert agents. The pH and exact concentration are adjusted according to routine skills in the art. See Goodman and Gilman's The Pharmacological Basis for Therapeutics (7^{th} Ed.).

As described above, the compositions of the present invention may be administered to a mammal. The examples set forth herein demonstrate use of the invention in cells in vitro. However, the results disclosed herein are transferable to any mammal of interest. For example, both the constitutively active CCK-BR receptor and the rat mu opioid receptor exhibit increased basal level activities in the absence of ligand (see FIG. 3, SMS-Luc results and FIG. 7), indicating that the constitutively active receptors are likely to generate a signal that mimics the normal, endogenous ligand-induced activity. In preferred embodiments, the compositions of the invention are used to treat humans. In addition, the compositions of the present invention may be used in veterinary medicine (e.g., to treat canines, felines, bovines, livestock, or zoo animals). One skilled in the art would recognize that any composition that is safe and effective in animals may also be administered to humans using similar dose parameters.

In preferred embodiments, the composition is administered to an individual in need of treatment (e.g., an individual diagnosed with a particular disease or disorder). In one preferred embodiment, nucleic acid delivery may be achieved by means of an accelerated particle gene transfer gun. The technique of accelerated particle gene delivery is based on the coating of nucleic acid to be delivered into cells onto extremely small carrier particles, which are designed to be small in relation to the cells sought to be transformed by the process. The nucleic acid encoding the desired gene sequence may be simply dried onto a small inert particle. The particle may be made of any inert material such as an inert metal (gold, silver, platinum, tungsten, etc.) or inert plastic (polystyrene, polypropylene, polycarbonate, etc.). Preferably, the particle is made of gold, platinum or tungsten. Most preferably the particle is made of gold. Gene guns are commercially available and well known in the art, for example, see U.S. Pat. Nos. 4,949,050; 5,120,657 (available from PowderJect Vaccines, Inc. Madison Wis.); or U.S. Pat. No. 5,149,655.

Alternatively, the composition described herein can be administered by any of a variety of routes including intravenously, (IV), intramuscularly (IM), intraperitoneal (IP), and subcutaneously. The inventive composition may be administered to mucosal surfaces by, for example, the nasal or oral (intragastric) route. Additionally, the composition may be administered using a suppository, transdermal patch, or alternatively by inhalation therapy.

Administration of the inventive compositions occurs in a manner compatible with the dosage formulation and in such amount as will be therapeutically effective. In the case of gene delivery, a dose formulation will be delivered in such amount that will produce an identifiable gene product (i.e., as detected directly (e.g., by ELISA) or by an assay for the biological activity of the gene product in the treated subject). The quantity of viral vector, or other gene delivery vehicle administered depends on the characteristics of the delivery vehicle and the characteristics of the subject to be treated. Precise amounts of the composition to be administered may depend on the judgment of the practitioner and may be particular to each subject and antigen. The dosage may also depend on the route of administration and will vary according to the size (i.e., weight) of the host. However, suitable dosage ranges are determined by one skilled in the art and may be of the order of 1 ng to 10 μg for naked DNA (e.g., if delivery of the nucleic acid is to occur via a gene gun) and 1 million to 1 billion plaque forming units (PFU) for other viral in vivo methods of nucleic acid delivery.

Suitable dose regimes are also variable, but may include an initial administration followed by any number of subsequent administrations. For example, the composition may include a single dose schedule, or a multiple dose schedule in which a primary course of administration may be 1-10 separate doses, followed by additional administrations given at subsequent time intervals required to maintain expression of the constitutively active, hypersensitive, or nonfunctional receptor, for example, at a given interval of months or years for a second administration, and if needed, a subsequent administration(s) after several months or years. Examples of suitable administration schedules include a monthly or bimonthly schedule, as long as the treatment is required (e.g., over a lifetime), or other schedules sufficient to maintain receptor expression to reduce or eliminate the disease symptoms or severity. Alternatively, the treatment of the present invention can be administered to achieve prevention of a particular disease or condition, or increased health (e.g., improved physiology, increased life span etc.). One important factor that governs the administration schedule is the amount of time that the receptor is expressed in the tissue. The dosage and administration procedure used in mice and other animal models can be scaled to humans or other animals by one skilled in the art.

Monitoring Expression

Successful expression of the constitutively active, hypersensitive, or nonfunctional receptor polypeptides of the invention in a cell or tissue can be assessed by standard immunological assays, for example the ELISA (see, Ausubel et al., Current Protocols in Molecular Biology, Greene Publishing Associates, New York, V. 1-3, 2000; Harlow and Lane, Antibodies, A Laboratory Manual, Cold Spring Harbor Laboratory, 1988, incorporated herein by reference).

Alternatively, the biological activity of the gene product of interest can be measured directly by the appropriate assay, for example, the assays provided herein. The skilled artisan would be able to select and successfully carry out the appropriate assay to assess the biological activity of the gene product of interest in a particular sample. Such assays (e.g., radioligand binding or receptor signaling assays) might require removing a sample (e.g., cells or tissue) from the individual to use in the assay. Expression of the particular receptor may be monitored by any of a variety of immunodetection methods available in the art. For example, the receptor may be detected directly using an antibody directed to the receptor itself or an antibody directed to an epitope tag (e.g., a FLAG tag) that has been included on the receptor for facile detection.

Kits

The present invention also provides therapeutic kits that are useful for carrying out the present invention. In one preferred embodiment, the kit provides a composition for in vitro administration of nucleic acids encoding constitutively active, hypersensitive, or nonfunctional receptors. In another preferred embodiment, the kit provides nucleic acid molecules encoding constitutively active, hypersensitive, or nonfunctional receptors that can be administered to a mammal. Preferably, the nucleic acid molecule is a viral or non-viral vector encoding a constitutively active, hypersensitive, or nonfunctional receptor. In certain preferred embodiments, the viral or non-viral vector includes cell specific ligands useful for targeting specific cell-types in a mammal.

According to the present invention, the kits contain nucleic acid molecules that may be administered by any method available in the art. In one preferred embodiment, the kits include a first container means containing a nucleic acid encoding a constitutively active, hypersensitive, or nonfunctional receptor, e.g., a viral or non-viral vector, in a pharmaceutically acceptable carrier. In one particularly preferred embodiment, the kits include an adenoviral vector encoding a constitutively active receptor, e.g., a constitutively active mu opioid receptor. Alternatively, if the means of delivery is a gene gun, the kit may include an aliquot of frozen or lyophilized nucleic acid encoding the constitutively active receptor. For gene gun delivery, the kit may also include a second container means that contains the small, inert, dense particles in dry powder form or suspended in 100% ethanol and, optionally, a third container means that contains the coating solution or the premixed, premeasured dry components of the coating solution. These container means can be made of glass, plastic, or foil and can be a vial, bottle, pouch, tube, bag, etc. The kit may also contain written instructions, such as procedures for administering the composition, or analytical information, such as the amount of reagent (e.g. moles or mass of nucleic acid). The written information may be located on any of the first, second, and/or third container means, and/or a separate sheet included, along with the first, second, and third container means, in a fourth container means. The fourth container means may be, e.g., a box or a bag and may contain the first, second, and third container means. It will be appreciated that this kit can be modified to include any reagent for administration described above, or known in the art.

All references cited herein are hereby incorporated by reference.

### EXAMPLES

The present invention can be further understood through consideration of the following non-limiting examples.

### Example 1

Constitutively Active Mu Opioid Receptor

This example describes the identification of a novel constitutively active rat mu opioid receptor and use of nucleic acids encoding this receptor in gene therapy.

Identifying Regions of Homology in the Mu Opioid Receptor

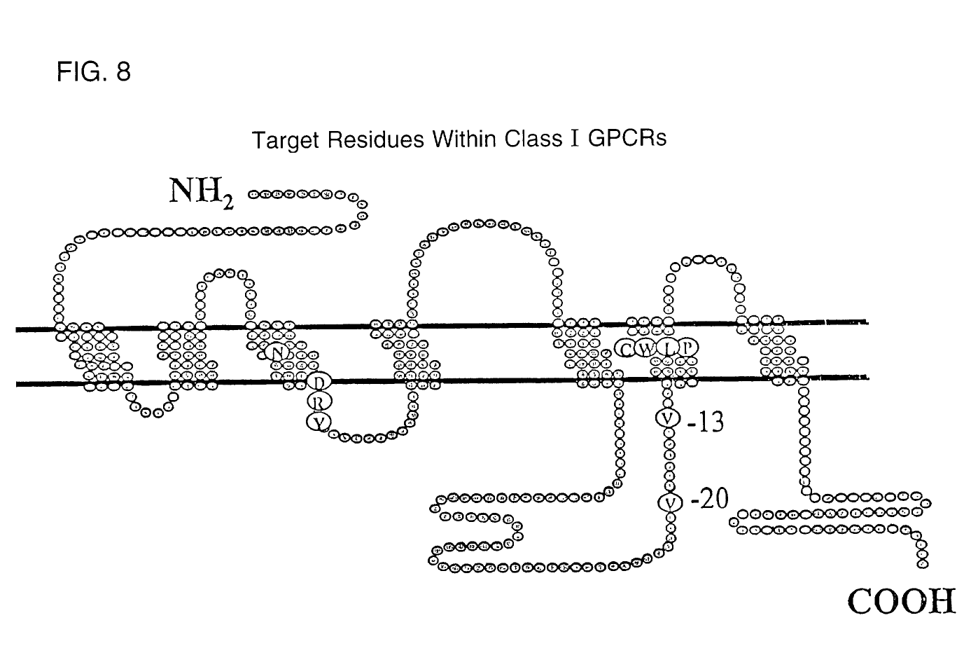

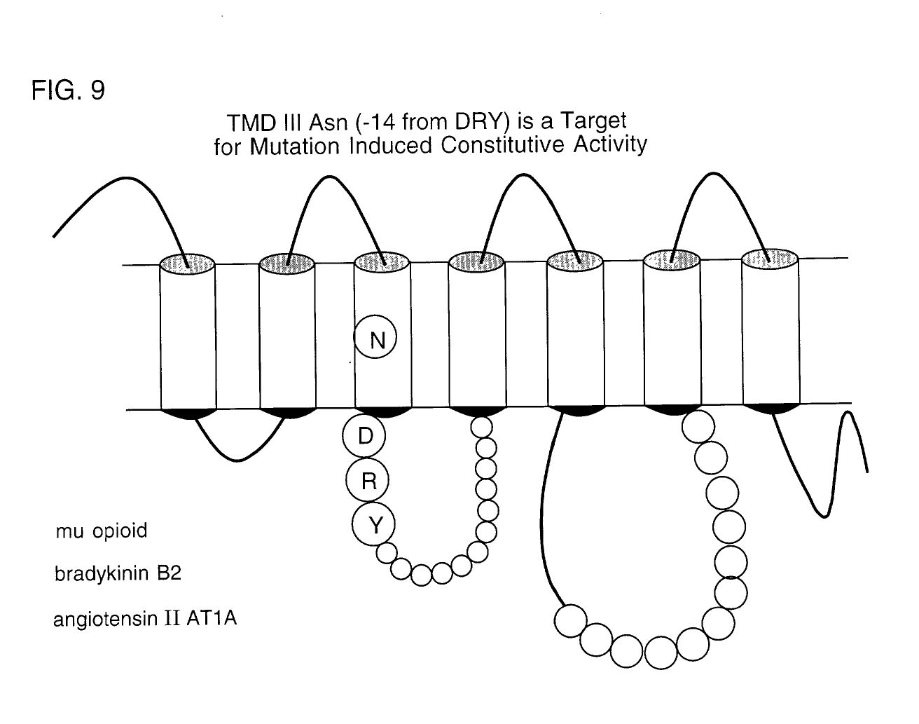

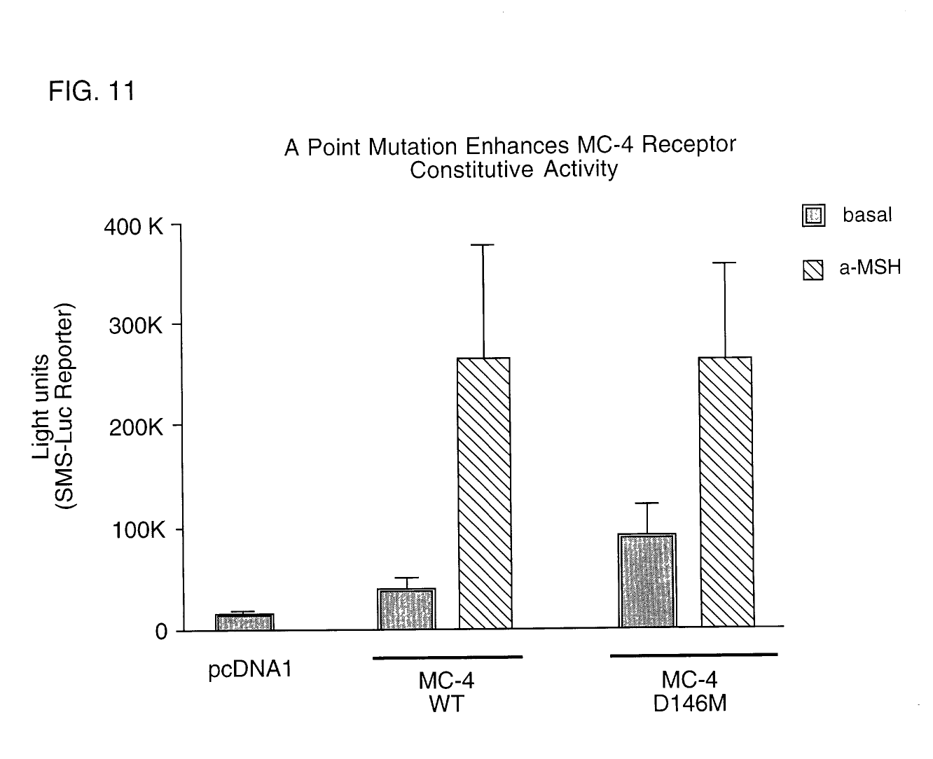

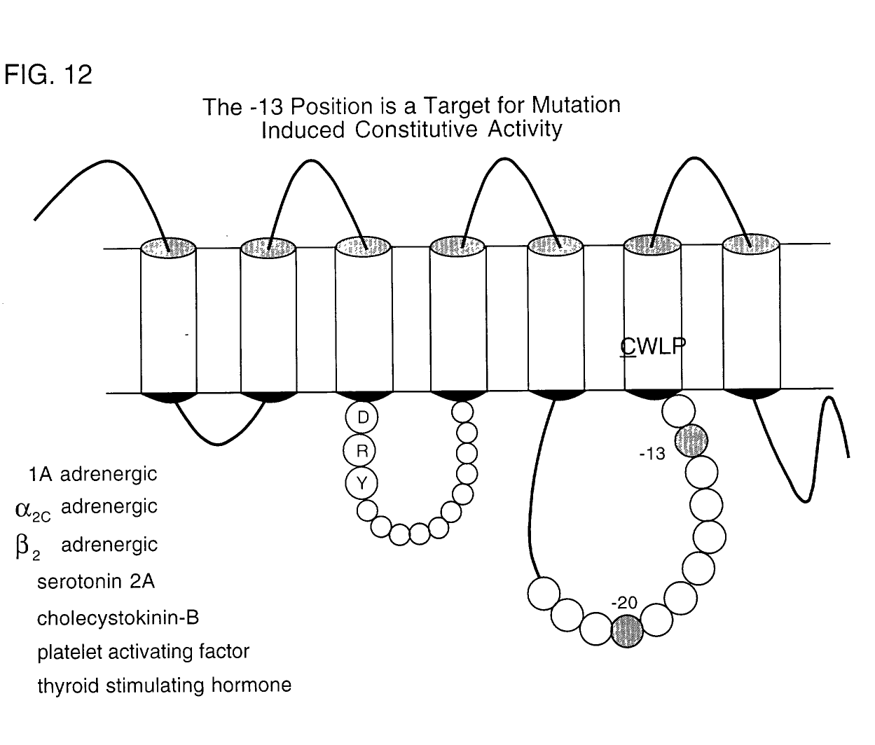

A database containing sequence information for known constitutively active Class A G protein-coupled receptors was generated by compiling available information from the prior art (see FIG. 1). The database was then used to identify key residues within Class A G protein-coupled receptors that are important for constitutive activity. These highly conserved residues are illustrated in FIG. 8. Of particular interest was the Asn residue at position 150 of SEQ ID NO: 1 in transmembrane domain III, which is conserved between the rat mu opioid receptor, the bradykinin B2 receptor, and the angiotensin II AT1A receptor (see FIG. 8). The ‘DRY’ motif at position 164-166 of SEQ ID NO: 1 is conserved between the oxytocin receptor, the vasopressin-V2 receptor, the cholecystokinin-A (CCK-A) receptor, the melanocortin-4 (MC-4) receptor, and the α_{1B} adrenergic receptor (see FIG. 9). It is important to note that this general motif, although not necessarily consisting of the specific residues ‘DRY’ (an alternative is, e.g., ‘ERY’), is conserved among all class A G protein-coupled receptors. In addition, the position corresponding to 13 residues N-terminal to the ‘CWLP’ motif is functionally conserved between the 1 A adrenergic receptor, the α2C adrenergic receptor, the β2 adrenergic receptor, the CCK-B receptor, the platelet activating factor receptor, and the thyroid stimulating hormone receptor (see FIG. 11) in that mutation of the amino acid at position −13 in each of these receptors results in constitutive activity. “Functionally conserved” means that the same amino acids are not necessarily present, but mutations in homologous or surrounding positions can result in constitutive activity.

Generating Mutant Mu Opioid Receptors

Based on the homology between the mu opioid receptor, the bradykinin B2, and the angiotensin II AT1A receptors at the Asn residue at position 150 of SEQ ID NO: 1, we chose to generate a rat mu opioid receptor having a point mutation at this position. An Asn150Ala mutation was introduced into the rat mu opioid receptor using standard molecular biological techniques. This mutant gene was then subcloned into expression vector pcDNA1 (Sambrook et al. supra).

Assaying Mutant Mu Opioid Receptors for Constitutive Activity

Reagents & Solutions: The cell culture media used in the assays described below was Gibco BRL #12100-046. This media was made according to manufacturer's recipe, pH adjusted to 7.2, filtered (0.22 micron pore), and supplemented with 1% Pen/Strep (Gibco #15140-122; 100% penicillin G 10,000 units/ml, and streptomycin 10,000 μg/ml) and 10% fetal bovine serum. Cell culture media lacking 10% fetal bovine serum was also generated. DNA used in the transfection experiments was purified and quantitated by measuring the absorbance at OD260. A LucLite Luciferase Assay Kit (Packard) was used to quantitate luciferase activity. Transfections were carried out using LipofectAMINE Reagent (Gibco #18324-012).

Constitutive activity of the Asn150Ala mutant rat mu opioid receptor was assessed using a luciferase assay. The rat mu opioid receptor is a Gαi coupled receptor. Therefore we chose to use the Gq5i reporter system, described in detail above (Broach and Thorner, supra), which switches the signaling pathway from Gαi to Gαq for reliable positive readout. HEK293 cells were transfected with the reporter construct SRE-Luc, an expression vector containing nucleic acid encoding Gq5i (Broach and Thorner, supra), and an expression vector containing nucleic acid encoding either the wild-type or the Asn150Ala mutant rat mu opioid receptor. Basal and ligand-stimulated luciferase activity was measured. The ligand used in this assay was [D—Ala²—MePhe⁴, Gly-ol⁵]enkephalin] (DAMGO). As a negative control, HEK293 cells were transfected with pcDNA1 (empty vector DNA), SRE-Luc, and the expression vector containing nucleic acid encoding Gq5i (Broach and Thorner, supra).

The luciferase assay was carried out as follows. On day 1, HEK293 cells in a T75 flask were washed with 15 ml serum-free media (or PBS), trypsinized with 5 ml 0.05% trypsin-EDTA (Gibco #25300-062), incubated at 37° C. for 3 minutes at which time 6-7 ml complete HEK293 media (Gibco #12100-046) and 10% Fetal Bovine Serum (Intergen #1050-90) were added. Thereafter, cells were collected in 50 ml centrifuge tubes, pelleted at 800-900 rpm (RCF˜275), and resuspend in 20 ml complete media. The cells were counted using a haemocytometer and diluted to 85,000 cells/ml in complete media. Using a repeat pipettor or cell plater, 100 μl of cells were added to each well of a Primaria 96-well plate (Falcon #353872). Cells were then incubated at 37° C., 5% CO₂ until use at 48 hours.

On day 3, cells were transfected using LipofectAMINE™ according to the manufacturer's protocol (Gibco #18324-012, Rockville, Md.).

On day 4, cells were stimulated as follows. Ligands for the receptor, either DAMGO or a non-peptide ligand (e.g., naloxone or naltrexone), were diluted to a desired concentration in serum-free media containing 0.15 mM PMSF (or other protease inhibitor(s)). The transfection media was then completely removed from cells and 50-100 μl stimulation media (i.e., media containing candidate ligands or the corresponding ligand free solvent) was added to each well. The cells were incubated for the desired time (standard is overnight) at 37° C., 5% CO₂, although the optimal stimulation time may vary depending on the particular receptor used. The optimal incubation time may be determined systematically by testing a range of incubation times and determining which one yields the highest level of stimulation. For concomitant assessment of two ligands (e.g., ligand induced inhibition of forskolin stimulated CRE activity) each stimulus is prepared at two times the desired final concentration and mixed in equal volumes prior to addition to cells.

On day 5, an assay for luciferase expression was carried out according to the manufacturer's instructions (Packard, Meridin, Conn.)

Results: Mu Opioid Receptor

Mutation of the Asn residue at position 150 of SEQ ID NO: 1 to Ala yielded a constitutively active rat mu opioid receptor. In FIG. 6 and Table 1, below, the results of the wild-type and Asn150Ala mutant rat mu opioid receptors are compared side by side. Shown in FIG. 6 are the basal and ligand-stimulated activities of the wild-type rat mu opioid receptor and the basal activity of the negative control vector (pcDNA 1 lacking any encoded gene). The basal activity of the wild-type rat mu opioid receptor is exceeded by the basal activity of the negative control vector. There is a significant increase (approximately 6.5 fold) in basal activity of the Asn150Ala mutant mu opioid receptor, indicating that the mutant mu opioid receptor is constitutively active.

TABLE 1

| | | Average Ligand |
|---|---|---|
| | Average Basal Activity | Stimulated Activity |
| Receptor | (Light Emission) | (Light Emission) |
| pcDNA 1 | 16,041 | 16,746 |
| (SRE + Gq5i) | | |
| wild-type rat mu opioid | 8,436 | 87,461 |
| receptor | | |
| (SRE + Gq5i) | | |
| Asn150Ala rat mu opioid | *56,498 | 86,996 |
| receptor | | |
| (SRE + Gq5i) | | |

*6.5-fold stimulation of basal level activity.

Gene Therapy Using Mu Opioid Receptor Nucleic Acid

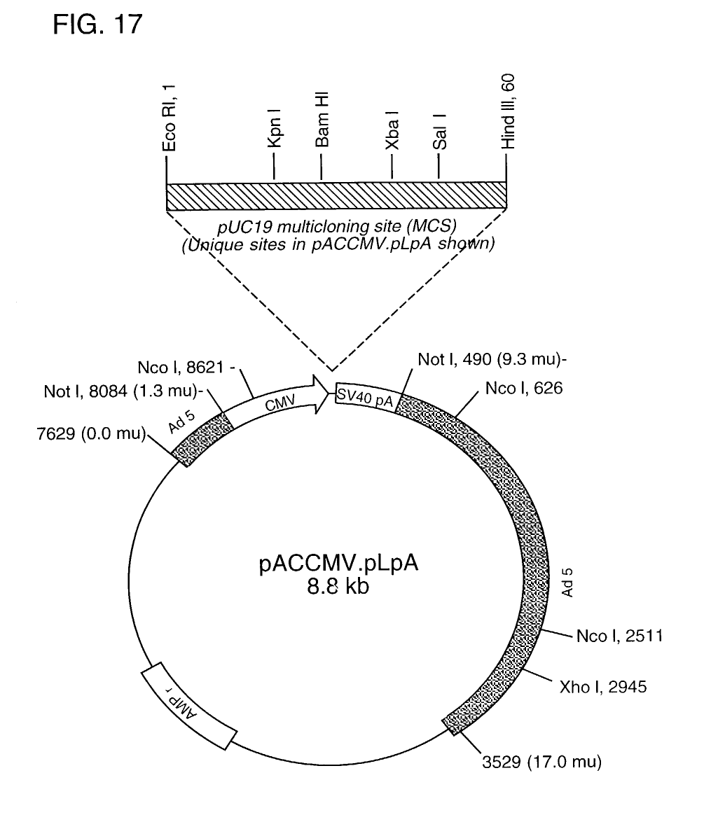

In a preferred gene therapy approach, an adenoviral construct is generated encoding the constitutively active (Asn150Ala) rat mu opioid receptor (see FIG. 17). The construct is next injected into the intrathecal space of rats. After 1-2 days, allowing for expression of the receptor in the rat spinal cord, tail flick experiments are carried out, as described, for example, in Pollack et al. (Pharm. Res. 17(6):749-53 (2000)). The tail flick response to radiant heat (the amount of time it takes for the rat to remove its tail from a heat source) determines the analgesic effect of the constitutively active mu opioid receptor. Constitutively active mu opioid receptors that reduce the sensitivity of a rat tail to heat are considered useful gene therapy constructs.

In humans, gene therapeutic agents containing nucleic acids encoding the constitutively active Asn150Ala human or rat mu opioid receptor may be injected into a patient for treatment of pain. Expression and activity of the constitutively active mu opioid receptor is assessed using well known methods, as described herein (e.g., standard immunological assays). Preferably, the expression and activity of the constitutively active rat mu opioid receptor is first examined in cells in vitro that are of the same type of cell or are the same cells (e.g., taken from the in vivo site and cultured in vitro), as those of the in vivo site. The gene therapeutic agent encoding the constitutively active rat mu opioid receptor is then injected into a patient that is experiencing pain, for example, the intrathecal space for treatment of back pain.

### Example 2

Constitutively Active Melanocortin-4 Receptor

This example describes the identification of a constitutively active melanocortin-4 (MC-4) receptor and use of such nucleic acids in gene therapy.

Identifying Regions of Homology and Generating MC-4 Receptor Mutants

As shown in FIG. 9, the “DRY” motif is conserved between the Class A G protein-coupled, oxytocin, vasopressin-V-2, cholecystokinin-A (CCK-A), melanocortin-4 (MC-4), and α_{1B} adrenergic receptors (FIG. 9). Based on this homology, plus precedent that substitution of aspartic acid within the DRY motif results in constitutively active oxytocin, vasopressin V-2, CCK-A, and α1B receptors, we hypothesized that substitution of the D (Asp) residue at position 146 of MC-4 by a non-charged residue would yield a constitutively active receptor (the MC-4 sequence is available as Genebank Accession is L08603). An Asp146Met mutant MC-4 receptor was generated using routine methods.

Assaying of Mutant MC-4 Receptors for Constitutive Activity

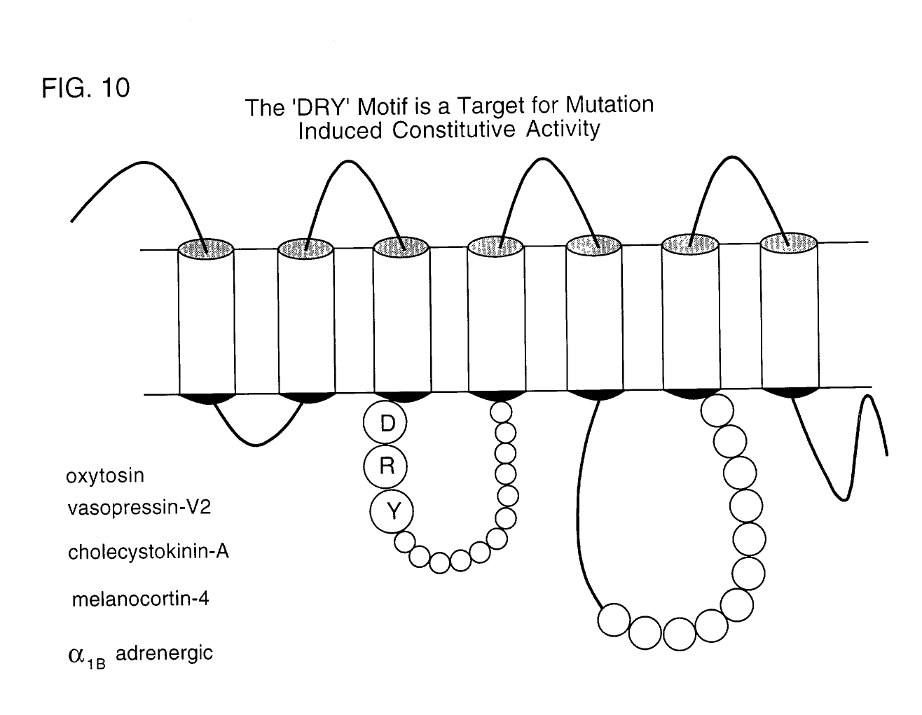

As demonstrated in FIG. 10, the reporter system assay was capable of detecting constitutive activity of the mutant Asp146Met MC-4 receptor. Briefly, HEK293 cells were cotransfected, as described above, with an expression vector encoding either the wild-type MC-4 receptor or the Asp146Met mutant MC-4 receptor and the reporter construct, SMS-Luc. As a negative control, cells were transfected with SMS-Luc and pcDNA1. Basal and ligand (αMHS) induced activity of the negative control, the wild-type MC-4 receptor, and the Asp146Met mutant MC-4 receptor were measured using the luciferase assay described above. The Asp146Met mutant MC-4 receptor mutant clearly exhibited a higher basal level activity than its wild-type counterpart.

Gene Therapy Using MC-4 Receptor Nucleic Acid

The MC-4 receptor is a G protein-coupled seven transmembrane receptor expressed in the brain that has been implicated in a maturity onset obesity syndrome associated with hyperphagia, hyperinsulinemia, and hyperglycemia in mice (Huszar et al. supra). Specifically, chronic antagonism of the MC-4 receptor by the agouti polypeptide induces a novel signaling pathway that increases glucose tolerance and results in increased body weight. Agonists that activate this pathway through the MC-4 receptor have been shown to be useful in decreasing body weight. Thus, according to the invention, nucleic acids encoding constitutively active MC-4 receptors are administered to a mammal to decrease glucose tolerance for treatment of obesity related to hyperphagia, hyperinsulinemia, and hyperglycemia. Gene therapy agents including nucleic acids encoding constitutively active MC-4 receptors are generated using any art available method and administered to the brain for treatment and/or management of obesity.

### Example 3

Constitutively Active β2 Adrenergic Receptors

This example describes the identification of constitutively active β2 adrenergic receptors and use of such nucleic acids in gene therapy.

Identifying Regions of Homology and Generating Constitutively Active β2 Adrenergic Receptor

As described in Samama et al. J. Biol. Chem. 268(7):4625-4636 (1993), a constitutively active mutant of the β2 adrenergic receptor was generated by replacing the C-terminal portion of the third intracellular loop of the β2 adrenergic receptor with the homologous region of the 1B adrenergic receptor (FIG. 1, page 3). This conservative substitution led to agonist independent activation of the β2 adrenergic receptor. In addition, the constitutively active receptor has an increased intrinsic affinity for β2 adrenergic receptor agonists and partial agonists, as well as an increased potency, and are therefore also hypersensitive.

Gene Therapy Using β2 Adrenergic Receptor Nucleic Acid

Agonists to the β2 adrenergic receptor have been widely used to treat asthma. In fact, inhaled beta-adrenergic agonists are the most commonly used treatments for asthma today (Drazen et al., Am. J. Respir. Care Critical Med. 162(1):75-80 (2000)). In addition, polymorphisms in the gene encoding the β2 adrenergic receptor have been identified and correlated with asthma severity (Holloway et al., Clin. Exp. Allergy 30(8):1097-103 (2000)). Thus, according to the present invention, constitutively active β2 adrenergic receptors are useful therapeutic agents in the treatment and prevention of asthma.

The constitutively active β2 receptors, described above, are provided on page 3 of FIG. 1. Thus, for treatment of asthma, nucleic acids encoding a constitutively active β2 adrenergic receptor are administered to the bronchial surface of a mammal, for example, via an inhaler. Gene therapy agents including nucleic acids encoding constitutively active β2 adrenergic receptors are generated using any art available method and administered to surfaces of the respiratory system for treatment and/or management of asthma.

### Example 4

Constitutively Active α1 Adrenergic Receptors

This example describes the identification of constitutively active α1 adrenergic receptors and the use of such nucleic acids in gene therapy.

Identification of Constitutively Active α1 Adrenergic Receptors

As illustrated in FIG. 1, page 2, numerous α1 adrenergic receptors have been identified that have constitutive activity. Indeed, nineteen different amino acid substitutions of the Ala at position 293 of the α1 adrenergic receptor result in constitutive activity of the receptor (Kjelsberg et al., J. Biol. Chem. 267(3):1430-1433 (1992)). Additional constitutively active mutants of the α1 adrenergic receptor include mutants of the DRY motif at the junction between transmembrane domain III and intracellular loop 2. These mutants include the Asp142Ala mutant (Scheer et al., Mol. Pharm. 57(2):219-231 (2000)) and the Arg143Lys mutant (Scheer et al., Proc. Natl. Acad. Sci USA 94(3):808-813 (1997)). Another constitutively active mutant of the α1 adrenergic receptor is the Asn63Ala mutant (Scheer et al., supra (1997)). Mutation of this conserved Asn63 residue located N-terminal to the DRY motif frequently leads to constitutive activity in a variety of other G-protein-coupled receptors (see FIG. 8). Other constitutively active α1 adrenergic receptors include the Cys128Phe mutant (in transmembrane domain III) (Perez et al., Mol. Pharmacol. 49(1): 112-122 (1996)); the Ala293Glu mutant (carboxyl end of IC3) (Perez et al., supra); and the Ala204Val mutant (transmembrane domain V) (Hwa et al., Biochemistry 36(3):633-639 (1997). Other mutants include those described in Allen et al. (Proc. Natl. Acad. Sci. USA 88(24): 11354-11358 (1991) and shown in FIG. 1, page 2).

Gene Therapy Using α1 Adrenergic Receptor Nucleic Acid

Phenylepinepherine is a commonly used agonist of the α1 adrenergic receptor for the treatment of nasal congestion. Thus, according to the present invention, constitutively active α1 adrenergic receptors are useful treatments for nasal congestion. Nucleic acids encoding constitutively active α1 adrenergic receptors can be administered, e.g., to the surfaces of nasal passages, e.g., via a nasal spray, as a nasal decongestant.

### Example 5

Constitutively Active and Nonfunctional Angiotensin Receptors

This example describes the identification of a constitutively active angiotensin receptor, as adopted from Groblewski et al. (J. Biol. Chem. 272:1822-1826 (1994)), and use of nucleic acids encoding a constitutively active and nonfunctional angiotensin receptor in gene therapy.

Identifying Regions of Homology and Generating

A constitutively active mutant of the AT1A angiotensin II receptor, a G protein-coupled receptor, was identified as described by Groblewski et al. (J. Biol. Chem. 272:1822-1826 (1994); Feng et al., Biochemistry 37(45):15791-15798 (1998); see also Feng et al. supra). Briefly, a previous molecular modeling study by Joseph et al. (J. Protein Chem. 14:381-398 (1995)) predicted an interaction between Asn 111 in transmembrane domain III and Tyr 292 of transmembrane domain VII in a non-activated AT1A angiotensin II receptor. Joseph et al. (supra) further predicted that in the activated receptor, this interaction would be disrupted. Groblewski et al. (supra) observed that the Asn 111 residue of the AT1A angiotensin II receptor is found at a homologous position in other peptide hormone receptors, including angiotensin 2 and Xenopus angiotensin, bradykinin, opioid, interleukin 8, and somatostatin receptor. In these receptors, mutation of the Asn 111 residue may yield constitutively active receptors. Furthermore, Groblewski et al. (supra) observed that mutation of Cys128 (Perez et al., Mol. Pharmacol. 49:112-122 (1996)) in the α-1B adrenergic receptor, which occupies a position homologous to that of Asn 111 in the AT1A angiotensin receptor, also induced constitutive activation. Assessment of constitutive activity in the corresponding AT1A receptor mutant was achieved by measuring and comparing the basal level of inositol phosphate production of the wild type and mutant angiotensin receptors (see Groblewski et al., supra Figs. 2, 3, and 4, supra).

In summary, through mutational analysis of the AT1A angiotensin II receptor, Growblewski et al. showed that mutation of the Asn at position 111 to Ala resulted in a receptor with strong constitutive activity. It will be appreciated that additional constitutively active AT1A angiotensin II receptors are identified by repeating the steps of identifying regions of homology, introducing mutations, and assaying for increased basal activity.

Gene Therapy Using Angiotensin Receptor Nucleic Acid

There are three angiotensin receptor subtypes, the angiotensin receptor I, II, and IV. The cardiovascular and other effects of the ligand angiotensin II are mediated by the angiotensin I and II receptors, which are seven transmembrane glycoproteins with 30% sequence similarity. The angiotensin I receptor plays a key role in cardiovascular homeostasis, whereas the angiotensin II receptor contributes to blood pressure and renal function. The function of the angiotensin IV receptor is unknown, but high levels of angiotensin IV receptor are found in the brain and kidney. (See De Gasparo et al., Pharmacological Reviews 52:415-472 (2000)).

Based on the role of angiotensin I and II in blood pressure regulation, specifically in increasing blood pressure (Sosa-Canache et al., J. Human Hypertension April; 14 Suppl; 1:S40-6 (2000); Siragy et al. Hypertension 35(5):1074-1047) (2000); Ackerman et al., Am. J. Physil. Regul. Integ. Comp. Physiol. 278(6):R1441-5 (2000)), mutants of angiotensin I and/or II receptors are useful therapeutics for disorders involving blood pressure, e.g., to raise or lower blood pressure. According to the invention, administration of nucleic acids encoding mutant angiotensin I or II receptors are used to raise or lower blood pressure, e.g., for treatment of hypertension or hypotension. For example, for treatment of hypertension, e.g., to lower blood pressure, a nucleic acid encoding a non functional angiotensin I or II receptor is selected. For treatment of hypotension, e.g., to raise blood pressure, constitutively active angiotensin receptor is selected. Treatment is achieved, for example, by injecting a nucleic acid encoding the non functional or constitutively active into the heart of a patient with hypertension or hypotension, respectively.

### Example 6

Constitutively Active Pituitar Adenylate Cyclase Activating Polypeptide Type I Receptor

This example describes the identification of a constitutively active pituitary adenylate cyclase activating polypeptide receptor, as adopted from Cao et al. (supra), and use of nucleic acids encoding a constitutively active pituitary adenylate cyclase activating polypeptide receptor in gene therapy.

Identifying Regions of Homology and Generating Constitutively Active Mutants of the Pituitary Adenylate Cyclase Activating Polypeptide Receptor

Pituitary adenylate cyclase activating polypeptide (PACAP) receptors belong to a family of Class B G protein-coupled receptors. The receptors of this family all couple to Gs or Gq to stimulate adenylate cyclase. Other peptide hormone receptors in this family include receptors for secretin, glucagon, glucagon-like peptide 1, growth hormone releasing hormone, gastric inhibitory peptide, parathyroid hormone, and calcitonin.

An analysis of amino acid sequence homology among the various receptors of Class B G protein-coupled receptors was carried out by Cao et al. (supra). The alignment revealed a highly conserved glutamic acid in the putative center of a second intracellular loop of the PACAP receptor (Cao et al., Fig. 1, supra). Mutant receptors, wherein the glutamic acid residue was altered, were assayed for constitutive activity. Specifically, basal level cAMP production was measured in cells expressing wild type or mutant PACAP receptors to identify constitutively active mutants (Cao et al. Fig. 4, supra). All mutations introduced at this position yielded constitutively active PACAP receptors (Cao et al. (supra)). Since the glutamic acid residue is highly conserved, this position is a target for mutation and analysis for other Class B G protein-coupled receptors.

Gene Therapy Using PACAP Nucleic Acid

PACAP is a neuropeptide originally isolated from ovine hypothalamus tissue and is one of the most potent known stimulators of adenylate cyclase. PACAP functions as a hypophysiotropic hormone and as a neurotransmitter, neuromodulator, and neurotrophic factor in the central nervous system. In light of these activities, gene therapeutic agents including nucleic acids encoding the PACAP receptor are useful in the treatment of a wide variety of biochemical and neurological conditions.

### Example 7

Constitutively Active Parathyroid Hormone Receptor

This example describes the identification of a constitutively active parathyroid hormone receptor, as adopted in part from Schipani et al. (New Engl. J. Med. 335:(10)708-714 (1996)), and use of nucleic acids encoding a constitutively active parathyroid hormone receptor in gene therapy.

Generating Constitutively Active Parathyroid Hormone Receptor

The parathyroid hormone (PTH) receptor is a Class B G protein-coupled receptor that couples independently to the adenylate cyclase-activating Gs protein and the PLCβ-activating Gq protein. In osteoblasts and osteoblast precursors, the PTH receptor couples to Gs to activate the adenylate cyclase-cAMP dependent protein kinase mechanism and to Gq to activate the phospholipase Cβ (PLCβ)-Ca²⁺/protein kinase C (PKC) second messenger signaling pathways (Whitfield et al., TiPS 16:382-386 (1995)). Other members of this gene family, which are highly conserved, include the receptors for calcitonin, secretin, growth hormone-releasing hormones, vasoactive intestinal polypeptide (types 1 and 2), gastric-inhibitory polypeptide, glucagon-like peptide 1, glucagon corticotropin-releasing factor, and the pituitary adenylate cylase activating polypeptide (Jüppner et al. Curr. Opin. Nephrol. Hypertens. 3(4):371-378, Fig. 1, p 373 (1994)).

Two polymorphic constitutively active PTH receptors have been identified (Schipani et al. supra). Briefly, upon comparison to wild-type sequence isolated from healthy patients, mutations in the gene encoding the PTH receptor were identified in patients with Janen's metaphyseal chondrodysplasia, a rare form of short-limbed dwarfism associated with hypercalcinmea and normal or low serum concentrations of PTH and PTH-related peptide (PTHrP). These mutations include a His223Arg mutation and a Thr410Pro missense mutation (Schipani et al. (Abstract) supra). In COS-7 cells expressing the mutant PTH receptors, basal cyclic AMP accumulation was four to six times higher than in cells expressing wild-type receptors (Schipani et al., see Fig. 4, supra). Other constitutively active Class B receptors can be identified using the sequence alignment provided by Jüppner et al. (supra) when residues that are homologous to H225 and T410 in the PTH receptor are targeted for mutation. The cAMP accumulation assay described by Schipani et al. (supra) is then employed to assess the basal activity of the mutant and the wild-type Class B receptor to determine whether the mutant receptor is constitutively active.

Gene Therapy Using PTH Receptor Nucleic Acid

The PTH receptor is known to trigger bone growth. Specifically, the PTH receptor triggers bone growth through cAMP-mediated production and secretion of autocrine and paracrine factors, such as insulin-like grown factor 1 and insulin-like growth factor-binding protein 5, which stimulate osteoblast precursor proliferation and production of bone constituents by mature osteoblasts (Whitfield et al. supra). Administration of PTH has been used to treat osteoporosis, frequently in combination with a therapy that prevents further bone loss (Whitfield et al. supra). According to the present invention, nucleic acids encoding a constitutively active or nonfunctional (inhibitory) PTH receptor are administered to the osteoclasts or osteoblast precursors of a patient for treatment of osteoporosis.

For example, nucleic acids encoding a constitutively active PTH receptor are injected directly into a bone of a patient at the site in the bone that has significant bone loss. Alternatively, osteoclasts or osteoblast precursor cells are transduced or transfected ex vivo and the cells later transferred to the site of bone loss in a patient diagnosed with osteoporosis. Alternatively, the cells are administered on a scaffolding and placed in the site of bone loss (see, e.g., WO 09/425,079; WO 09/012,603; WO 09/012,604; WO 09/409,760, incorporated herein by reference). In certain cases, it may be desirable to carry out the gene therapeutic treatment simultaneously with the administration of agents that inhibit bone loss, and such determination can be made by one skilled in the art. Of course, one skilled in the art will appreciate that the activity of the PTH receptor would have to be closely monitored, and possibly titrated, as described herein, due to the fact that a constitutively active form of the PTH receptor is associated with disease.

### Example 8

Constitutively Active Estrogen Receptor

This example describes the identification of a constitutively active estrogen receptor, as adopted from Weis et al., Molecular Endocrinology 10(11):1388-1398 (1996) and White et al., EMBO J. 16:1427-1435 (1997), and use of nucleic acids encoding a constitutively active estrogen receptor in gene therapy.

Identifying Regions of Homology and Generating Constitutively Active Estrogen Receptors

The estrogen receptor α (ER α or ER β) is a member of the nuclear steroid receptor superfamily that regulates the transcriptional activation of many important genes. Constitutively active ERαs have been identified, as described in Weis et al. (supra). Briefly, the role of Tyr 537 in the transcriptional response of the ERα was examined based on the fact that this residue is located close to a region of the hormone-binding domain previously shown to be important in hormone-dependent transcriptional activity, and further because this amino acid has been proposed to be a tyrosine kinase phosphorylation site important in the activity of the ERα (Weis et al. supra). It was shown that two of the ER α mutants, Tyr537Ala and Tyr537Ser, displayed estrogen-independent constitutive activity that was approximately 20% and 100% of the activity of the wild-type receptor, respectively. A reporter assay system was used to measure the ability of the wild-type and mutant ERα to activate transcription. Specifically, an estrogen responsive construct was made that included two estrogen-response elements, the pS2 gene promoter, and a chloramphenicol acetyl transferase (CAT) reporter gene (Weis et al., supra). A similar mutation of residue 541 in ERα (substitution with an amino acid with reduced hydrophobicity) also yielded a constitutively active ER (White et al., supra).

In order to identify additional constitutively active ERs, nonconstitutively active ER polypeptides are compared to other family member receptor polypeptides that are constitutively active. Regions of amino acids sharing homology are identified and targeted for mutation. These mutant ERs are then assessed using a reporter assay system, such as the CAT reporter assay system described by Weis et al. (supra) (or described herein) to determine whether they possess constitutive activity compared to their nonconstitutively active counterparts.

The above steps are exemplified by Tremblay et al. (Canc. Res. 58(5):877-81 (1998)). The amino acid sequences of ERα and ERβ were compared and regions of homology were identified between tyrosine 537 of Erα and tyrosine 443 of the nonconstitutively active ERβ (Tremblay et al. supra). These residues are known to be important for constitutive activity. To test whether constitutive activity could be conferred to ERβ, corresponding mutations were generated in the ERβ protein at tyrosine 443 (Tyr443Phe, Tyr443Ser, and Tyr443Asn). The resulting ERβ mutants were introduced by transient transfection into COS-1 cells and basal transcriptional activity measured using a luciferase reporter assay system responsive to ER activation (Tremblay et al. supra). The Tyr443Ser and Tyr443Asn ERβ mutants exhibited a basal level of transcriptional activity that equaled the ligand-stimulated level of transcriptional activity observed for the wild-type receptor (Tremblay et al. Fig. 1, supra). Thus, constitutive activity was successfully transferred to ERβ using systematic analysis.

Gene Therapy Using the Estrogen Receptor Nucleic Acid

It is well known that the amount of estrogen released by the ovaries decreases with the onset of menopause. Therefore, the symptoms of menopause are treated by administration of nucleic acid encoding a constitutively active estrogen receptor to the organs of the female reproductive system, e.g., the uterus or ovaries (e.g., using tissue specific administration and/or tissue specific promoters). For example, nucleic acids encoding a constitutively active estrogen receptor (e.g., naked DNA or nucleic acid contained in a viral vector) are injected directly into the uterus. Assessment of expression of the ER is carried out using standard immunological assays, as described herein. Of course, one skilled in the art will appreciate that the activity of the ER would have to be closely monitored, and possible titrated, as described herein, due to the fact that a constitutively active form of the ER is associated with breast cancer ((Tremblay et al., supra)).

### Example 9

Hypersensitive Erythropoietin Receptor

This example illustrates use of a hypersensitive EPO receptor for treatment of anemia using gene therapy.

Identifying Regions of Homology and Generating Hypersensitive Erythropoietin Receptors

The EPO receptor is a single transmembrane receptor that is a member of the cytokine receptor family. Hypersensitive EPO receptors are identified by comparing the amino acid sequences of family members of non-hypersensitive and hypersensitive receptors of the cytokine receptor family to identify regions of homology and target specific amino acid residues for mutation. Once identified, mutant EPO receptors are generated using standard molecular biological techniques, as described herein, and assayed for hypersensitivity.

One assay that can be employed in detecting a hypersensitive EPO receptor is an assay that monitors the Jak2/Stat5 signaling pathway. Upon activation of the EPO receptor, Jak2 associates with EPO receptor and undergoes autophosphorylation. The activated Jak2 subsequently phosphorylates both the EPO receptor and the transcription factor, Stat5. Activated Stat5 then translocates to the nucleus, recognizes a specific base sequence within the promoter of its target gene, and activates transcription of that gene. In light of these receptor-induced activities, screening mutant receptors for hypersensitivity is accomplished by assaying EPO receptor-dependent activation of Jak2 and Stat5 by immunoprecipitation and immunoblot analysis, as described in Watowich et al. (Blood 34:2530-2532 (1999)). This assay is carried out in cells expressing the mutant EPO receptor or the wild-type EPO receptor and the basal activities of these receptors compared. A mutant EPO receptor that exhibits an response to low doses of EPO (i.e., activation of Jak2 and Stat5) relative to cells expressing the wild-type EPO receptor, is identified as a hypersensitive EPO receptor.

Gene Therapy Using Erythropoietin Receptor Nucleic Acid

The EPO receptor is expressed almost exclusively on erythroid precursor cells and functions to control the development of red blood cells. Deficiencies in the transmission of the EPO receptor signaling cascade leads to clinically abnormal red blood cell production, and has been linked to a number of diseases including anemia.

Gene therapeutic agents including nucleic acids encoding hypersensitive EPO receptors are transfected into erythroid precursor cells ex vivo and administered to a patient for treatment of anemia (see, e.g., Sokolic et al. supra). For example, bone marrow cells are collected from a patient and transfected or transduced with nucleic acid encoding a hypersensitive EPO receptor (e.g., see Kauppinen et al. Mol Genet. Metab. 65(1):10-7 (1998)). Expression of the transgene can be monitored using immunoblot analysis and other well known methods. The cells, expressing the hypersensitive EPO receptor, are then readministered to the patient and allowed to proliferate in vivo. Alternatively, DNA encoding a hypersensitive EPO receptor can be injected directly into a patient, e.g., intravenously.

### Example 10

Constitutively Active Glucagon-like Peptide-1 Receptor

This example describes the use of nucleic acids encoding a constitutively active glucagon-like peptide-1 receptor in gene therapy.

The glucagon-like peptide-1 (GLP-1) receptor is a G protein-coupled receptor (Graziano et al. (Biochem. Biophys. Res. Commun. 196(1):141-146 (1993)). The human and rat GLP-1 receptor genes have been cloned and compared and regions of conservation identified (Dillon et al., Fig. 1, supra). GLP-1 receptor is activated by GLP-1, a hormone secreted from the distal gut that stimulates basal and glucose-induced insulin secretion and proinsulin gene expression (Dillon et al., supra). GLP-1 is associated with inhibition of upper gastrointestinal motility and involvement of the CNS (van Dijk et al., Neuropeptides 33(5):406-414 (1999)).

The involvement of the GLP-1 receptor in basal and glucose-induced insulin secretion and proinsulin gene expression is good evidence that nucleic acids encoding constitutively active GLP-1 receptors are useful in the treatment of diabetes. For example, B cells defective in glucose-dependent insulin secretion and production are isolated from a patient, cultured in vitro, and transfected with nucleic acid encoding a constitutively active GLP-1 receptor (e.g., a retroviral vector containing the nucleic acid encoding a constitutively active GLP-1 receptor). The transduced cells are then injected back into the patient intravenously for treatment of diabetes (see e.g., Sokolic et al., Blood 87(1):42-50 (1996)).

### Example 11

Constitutively Active and Nonfunctional Cholecystokinin-B/Gastrin Receptors (CCK-BR)

This example describes the identification of a constitutively active CCK-BR receptor, as adopted from Beinborn et al. (J. Biol. Chem. 273(23): 14146-14151 (1998) and Beinborn et al., Gastroenterology 110, (suppl.) A1059) (1996)), and use of nucleic acids encoding a constitutively active and nonfunctional CCK-BR in gene therapy.

Identifying Regions of Homology and Generating Mutant CCK-BR Receptors

Molecular characterization of the third intracellular loop of the human CCK-BR led to the identification of a point mutation (Leu325Glu) which results in constitutive CCK-BR activity (see, Beinborn et al. supra (1996)). Briefly, the strategy was based on the theory that domain swapping between related polypeptides with different second messenger couplings could yield receptors having increased basal activity. Segments of 4-5 amino acids were substituted in the third intracellular loop of the CCK-BR with corresponding sequences from the vasopressin 2 receptor, a protein with 30% amino acid identity to CCK-BR. However, these proteins are coupled to different signal transduction pathways. CCK-BR is coupled to phospholipase C activation, whereas the vasopressin 2 receptor is coupled to adenylyl cyclase as the predominant signal transduction pathway (Beinborn et al., supra (1996)).

Assaying Mutant CCK-BR Receptors for Constitutive Activity

As described in Beinborn et al., recombinant receptors were transiently expressed in COS-7 cells and ligand affinities were assessed by ¹²⁵I CCK-8 competition binding experiments. In addition, phospholipase C-mediated production of inositol phosphate was measured in the absence and in the presence of agonists. One of the block substitutions from the vasopressin 2 receptor, 250AHVSA, conferred agonist-independent constitutive activity when introduced into the corresponding region of the third intracellular loop of the CCK-BR. The mutant CCK-BR triggered a 10-fold higher basal turnover of inositol phosphate compared to wild-type CCK-BR. Substitution of 253SA and even 253S alone within the same segment was sufficient to confer constitutive activity as well (Beinborn et al., (Abstract) supra (1996).)

Additional studies were carried out as described in Beinborn et al. (supra (1998)). In particular, the Leu325Glu CCK-BR mutant triggers constitutive production of inositol phosphates to levels exceeding wild-type CCK-BR (Beinborn et al., Fig. 1A supra (1998)). Briefly, the human wild-type CCK-BR and the constitutively active Leu325Glu CCK-BR mutant were transiently expressed in COS-7 cells. Control cells (“no receptor”) were transfected with the empty expression vector, pcDNA1. Cells were pre-labeled overnight with myo-[³H]inositol and then stimulated with ligand for 30 to 60 minutes in the presence of 10 mM LiCl. The constitutively active CCK-BR mutant is clearly distinguished from the wild-type receptor by its ability to trigger inositol phosphate production in the absence of agonist.

In addition to these studies, we performed luciferase assays to measure the constitutive activity of the Leu325Glu CCK-BR mutant. HEK293 cells were transfected (as described above) with SMS-Luc and an expression vector encoding any one of pcDNA1, wild-type CCK-BR, or Leu325Glu CCK-BR. As demonstrated in the left panel of FIG. 3, the Leu325Glu CCK-BR mutant has increased basal level activity compared to the wild-type CCK-BR.

Gene Therapy Using CCK-BR Nucleic Acid

CCK-BR is a G protein-coupled receptor that has been implicated in modulating memory, anxiety, and pain perception, as well as in regulating gastrointestinal mucosal growth and secretion (Beinborn et al. supra (1998)). Thus, gene therapy treatment with nucleic acids encoding a constitutively active CCK-BR is applicable to the treatment of a wide range of diseases. These conditions may be treated with agonists or antagonists to achieve a desired outcome. For example, increasing memory is generally be treated with an agonist to the CCK-BR receptor, whereas the conditions of anxiety, pain perception, and gastrointestinal mucosal growth and secretion are generally treated with antagonists of the CCK-BR receptor.

This knowledge can be applied to determine the type of receptor administered to obtain the desired outcome. For example, since treatment of memory loss generally requires an agonist, nucleic acids encoding a constitutively active CCK-BR are administered to the brain for treatment of memory loss. Alternatively, since antagonists are generally administered for treatment involving anxiety, pain perception, and gastrointestinal mucosal growth and secretion, a nonfunctional (i.e., a dominant negative CCK-BR receptor) may be administered for treatment of these conditions, e.g., to the nervous system for treatment of anxiety, to a site where a mammal is experiencing pain for pain management, or to the gastrointestinal tract for treatment of gastrointestinal disorders, respectively. Nucleic acids encoding a constitutively active CCK-BR are generated using any art available method and administered to a mammal for the treatment of a disease or disorder, as described above.

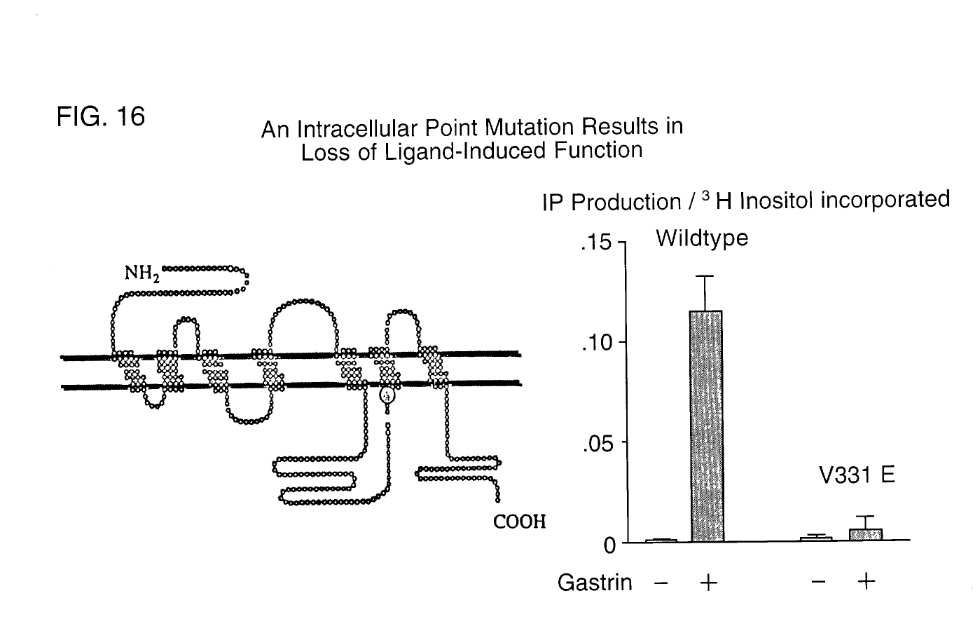

In one particular example, a nucleic acid encoding CCK-BR receptor having a point mutation that generates a receptor that displays normal ligand binding, but does not transmit a ligand induced signal issued as a gene therapeutic agent. Specifically, a Val1331Glu mutation in the CCK-BR receptor yields a hyposensitive receptor with little or no stimulation of inositol phosphate production, although the binding of the ligand to the receptor is normal (see FIG. 16). This nonfunctional receptor acts as a sink for endogenous ligand and effectively lowers the endogenous ligand concentration while blocking transmission of the ligand induced signal. In one gene therapeutic protocol, nucleic acid encoding this nonfunctional receptor is administered to the stomach to act as a sink for the gastrin ligand and thereby diminish gastrin-dependent acid secretion. Such administration can be used to treat peptic ulcer disease.

### Example 12

Nonfunctional Bradykinin Receptor

This example describes use of nucleic acids encoding a nonfunctional bradykinin receptor in gene therapy.

Gene Therapy Using Nucleic Acid Encoding the Bradykinin B2 Receptor

The bradykinin B1 and B2 receptors are members of a family of G protein-coupled receptors that respond to kinins, a family of biologically active peptides that produce a number of biological effects, including activation of sensory pain fibers, smooth muscle contraction, endothelium-dependent vasodilation, and plasma extravasation (Marie et al. supra (1999)). In addition, the bradykinin B2 receptor has been implicated in hypothyroidism (Savoie et al., Am. J. Physiol., 255(4 Pt. 1):E411-5 (1988)).

Given the number of diseases and conditions with which the bradykinin B2 receptor has been implicated, a wide variety of gene therapy treatments using nucleic acids encoding mutant bradykinin B2 receptors or other kinin receptors, can be envisioned. As but one example, since the bradykinin B2 receptor activates sensory pain fibers, a nucleic acid encoding a nonfunctional bradykinin B2 receptor is administered for the treatment of pain. Bradykinin B2 gene therapy agents are generated using any art available method and administered to a mammal for treatment of disease, as described above.

### Example 13

Nonfunctional CCR-3 Receptors

The CC chemokine (CCR-3) receptor is a seven-transmembrane-spanning G protein-coupled receptor expressed on thymocytes that plays a major role in the recruitment of inflammatory cells in an allergic response. Specifically, the CCR-3 receptor binds the polypeptide eotaxin to effect the regulation of eosinophil trafficking. Eosinophils are important players in the asthmatic response. Antagonists that inhibit this pathway through the CCR-3 receptor are useful therapeutic agents in the treatment and prevention of asthma. Thus, gene therapy agents that include nonfunctional receptors are preferred agents for treatment of asthma. Nonfunctional mutants of the CCR-3 receptor may be generated by referring to a database of conserved G protein-coupled receptors having mutations that make the receptors nonfunctional and mutating the CCR-3 receptor at homologous positions.

For treatment of asthma, nucleic acids encoding a nonfunctional CCR-3 receptor are administered to the bronchial surface of a mammal, for example, via an inhaler. Gene therapy agents including nucleic acids encoding nonfunctional CCR-3 receptors are generated using any art available method and administered to the brain for treatment and/or management of obesity.

### Example 14

Constitutively Active Dopamine Receptors

This example describes the use of nucleic acids encoding constitutively active dopamine receptors in gene therapy.

Mammalian dopamine receptors are seven transmembrane domain G protein-coupled proteins that fall into the class A or rhodopsin family based on conservation of amino acid sequence. Dopamine receptors can be further divided into two major types, D1-like and D2-like. These receptor groups are distinguished based on gene structure, signal transduction pathways, and sensitivity to class specific agonist and antagonist drugs (Emilien et al., Pharmacol. Ther. 84:133-156 (1999); Missale et al., Physiol. Rev. 78:189-225 (1998); Vallone et al., Neurosci. Biobehav. Rev. 24:125-132 (2000). The D1-like receptors include the D1 and D5 subtypes. These receptors are encoded by a single exon and signal primarily through Gs mediated activation of adenylate cyclase. The D2-like receptors include the D2, D3, and D4 subtypes. Each of the D2-like receptors is encoded by multiple exons offering the potential for alternatively spliced variants to exist. Dopamine-mediated signaling through the D2-like receptors is primarily through Gi/o induced inhibition of adenylate cyclase and modulation of ion channels.

The predominant dopamine receptors found in the striatum are the D1 and D2 subtypes (Emilien et al., Pharmacol. Ther. 84:133-156 (1999). Expression has been shown by in situ hybridization, immunohistochemistry, and receptor autoradiography. Although it is agreed that the D1 and D2 receptors are highly expressed in striatum, the degree to which there is coexpression of D1 and D2 receptors within individual striatal neurons remains controversial (Missale et al., Physiol. Rev. 78:189-225 (1998); Surmeier et al., J. Neurosci. 16:6579-6591 (1996); Aizman et al., Nat. Neurosci. 3:226-230 (2000). Many studies have suggested that D1 receptors are expressed on dynorphin/substance P neurons whereas D2 receptors appear preferentially expressed on enkephalin-producing cells. Others, using confocal microscopy and functional readouts (e.g. sodium channel activation) suggest there is coexpression of both the D1 and D2 receptors in many, if not all, striatal neurons.

It is quite likely that both striatal D1 and D2 receptors modulate locomotor function, and both are therefore useful targets for the development of therapeutics for Parkinson's disease (PD). Parkinson's disease affects about 1% of adults over the age of 60. The full clinical manifestations include bradykinesia, rigidity, tremor, and gait abnormalities. The disease results from degeneration of the dopaminergic nigrostriatal pathway. The trigger for the degenerative process in most cases remains unknown. A minority of cases results from genetic abnormalities (e.g. mutation in the alpha synuclein or the Parkin gene) (Rohan de Silva et al., Current Opinion in Genetics & Development 10:292-298 (2000). With the gradual loss of dopaminergic neurons in the substantia nigra, there is progressive damage to the axonal projections that innervate the striatum. The loss of nigrostriatal dopaminergic neurons leads to a decrease in dopamine mediated striatal signaling (Rohan de Silva et al., Current Opinion in Genetics & Development 10:292-298 (2000); Emilien et al., Pharmacol. Ther. 84:133-156 (1999); Missale et al., Physiol. Rev. 78:189-225 (1998). In humans as well as in rodents and nonhuman primates, toxins that destroy dopaminergic neurons (e.g. MPTP, 6-OH dopamine) result in the acute onset of Parkinsonian symptoms. Use of these toxins has enabled the development of animal models of PD.

Therapeutic strategies for PD are aimed at restoring dopaminergic activity in the striatum. One means to achieve this is to increase central dopamine levels. Levo-dopa (L-dopa), the precursor of dopamine has been the primary drug used for this purpose. When administered peripherally, L-dopa (unlike dopamine) crosses the blood brain barrier and is then enzymatically converted to dopamine. In patients with Parkinson's disease, loss of nigrostriatal presynaptic cells leads to dopamine depletion despite intact striatal postsynaptic neurons. With disease progression pharmacotherapy is ultimately insufficient to restore normal striatal dopaminergic signaling. In addition, L-dopa administration to patients with advanced PD results in dyskinesias and periods of marked fluctuation in motor activity (‘on-off effect’). Alleviation of these side effects has been a major challenge in the treatment of PD and has prompted a search for therapeutic strategies that can provide a sustained level of dopaminergic signaling.

One approach to restore striatal dopaminergic activity and at the same time to potentially avoid the consequences of long term L-dopa administration is through the introduction of constitutively active dopamine receptors. Accumulating evidence supports the idea that the D1 and D2 receptors act synergistically in mediating motor function (Emilien et al., Pharmacol. Ther. 84:133-156 (1999); Missale et al., Physiol. Rev. 78:189-225 (1998); Paul et al., J. Neurosci. 12:3729-3742 (1992); Usiello et al., Nature 408:199-203 (2000). Therefore, constitutively active dopamine receptors may be administered alone as well as in combination. In addition, these constitutively active dopamine receptors may be administered in conjunction with any other Parkinson's therapeutic including, without limitation, L-dopa, dopamine synthetic enzymes (for example, tyrosine hydroxylase or aromatic amino-dopacarboxylase), neuronal growth factors (for example, glial cell line-derived neurotrophic factor (GNDF)), or dopamine receptor agonists.

Expression of constitutively active dopamine receptors in the striatum provide a number of advantages for Parkinson's disease therapy. First, activated rather than wild type receptors are expressed. With constitutively active receptors, mutation-induced signaling persists even after dopamine depletion (as typically occurs with progression of Parkinson's disease). In addition, the use of constitutively active receptors as a therapy also provides a means to attain a stable level of striatal signaling and thus circumvent one of the major disadvantages of classical L-dopa treatment, the fluctuating motor responses and the dyskinesias which occur in patients with advanced disease. Moreover, the D2L and D1 receptors may be expressed individually or in combination. Ample evidence suggests D1 and the D2L receptors act synergistically in stimulating motor function (Emilien et al., Pharmacol. Ther. 84:133-156 (1999); Missale et al., Physiol. Rev. 78:189-225 (1998); Paul et al., J. Neurosci. 12:3729-3742 (1992); Usiello et al., Nature 408:199-203 (2000). In addition, recombinant adeno-associated virus (rAAV) may be utilized rather than adenovirus as a gene therapy vector. rAAV is less immunogenic than adenovirus and therefore persists for a considerably longer period of time. Adenovirus constructs used previously (Ikari et al., Brain Res. Mol. Brain Res. 34:315-320 (1995); Ingram et al., Mech. Ageing Dev. 116:77-93 (2000)) began to disappear 3-5 days after infection of the CNS. It is estimated that expression of rAAV should last a minimum of 60 days (Bjorklund et al., Brain Res. 886:82-98 (2000).

Constitutively Active Dopamine Receptors

It is well established that the D1 receptor is coupled to Gs mediated activation of adenylate cyclase, which in turn leads to elevation in cellular cAMP. D1R activation of Gs was confirmed using both the luciferase assay described herein as well as a cAMP radioimmunoassay. In contrast, D2 receptors (both long and short isoforms) are linked to G_{i/o} coupled pathways. Activation of the D2 receptor leads to alpha subunit-mediated inhibition of adenylate cyclase with a resultant decrease in cAMP (Emilien et al., Pharmacol. Ther. 84:133-156 (1999); Missale et al., Physiol. Rev. 78:189-225 (1998); Vallone et al., Neurosci. Biobehav. Rev. 24:125-132 (2000). Activation of G_{i/o} was also confirmed for the D2L and D2S receptors by expressing these receptors in HEK293 cells and measuring activity with the Gq5i/SRE luciferase reporter gene assay described above.

In addition to these major pathways, there is evidence that second messenger signaling linked to dopamine receptors includes certain other pathways that are highly cell type specific (Missale et al., Physiol. Rev. 78:189-225 (1998); Jiang et al., Proc. Natl. Acad. Sci. USA 98:3577-3582 (2001). Stimulation of dopamine receptors potentially results in activation of potassium channels, inhibition of calcium currents, and activation of mitogen activated protein kinase. In addition, in certain cellular milieus, both the D1 and D2 receptors have been shown to activate phospholipase C, leading to phosphatidylinositol-mediated increases in intracellular calcium.

Assays based on any of the above signaling pathways may be used to identify or confirm constitutive activity for a dopamine receptor simply by looking for increased activity relative to a wild-type control receptor, as described herein.

In particular, to isolate constitutive dopamine receptors, the relevant dopamine receptor cDNAs (e.g., D1, D2S, or D2L) are obtained or generated by PCR and preferably cloned into the expression vector, pcDNA1.1. Single stranded uracil template is then preferably used as the template for site-specific mutagenesis by standard techniques.

Potential amino acid targets for mutagenesis include two D1R (Cho et al., Mol. Pharmacol. 50:1338-1345 (1996); Charpentier et al., J. Biol. Chem. 271:28071-28076 (1996)) and one D2R (Wilson et al., J. Neurochem. 77:493-504 (2001)) point mutations reported to confer ligand independent signaling to the respective receptor. These may be generated as previously described (Beinborn et al., Nature 362:348-350 (1993); Kopin et al., J. Biol. Chem. 270:5019-5023 (1995)) and assessed by any of the assays described herein. These mutations, as characterized in the literature, confer only a minimal level of constitutive activity. Ideally, a basal level of signaling can be achieved which approximates >50% of the dopamine-stimulated maximum activity. To enhance activity, serial amino acid substitutions may be introduced in candidate locations. This approach produces receptors with a wide range of basal signaling including ones with marked constitutive activity (Kjelsberg et al., J. Biol. Chem. 267:1430-1433 (1992); Scheer et al., Proc. Natl. Acad. Sci. USA 94:808-813 (1997). An additional strategy, which may be used, is to introduce combinations of weakly activating mutations in an attempt to further increase basal signaling. Specific mutations that may be introduced into the dopamine 1 receptor include replacement in intracellular loop 3 of the amino acid −20 from the “CWLP” sequence with either an I, E, or S, or replacement in transmembrane region 6 of the L in the “CWLP” sequence with either an A, V, K, or E. Specific mutations that may be introduced into the dopamine 2 receptor include replacement in intracellular loop 3 of the amino acid −13 from the “CWLP” sequence with either an E, K, R, A, S, or C.

In addition, the deduced amino acid sequence of the D1 and D2 receptors include “hotspots” relative to conserved signature motifs (e.g., DRY) in other class A GPCRs. Additional mutants may be constructed based on this hotspot in intracellular loop II. For example, the D in the “DRY” sequence may be replaced with either an M, T, V, I, or A, or the R may be replaced with either an A or K. As above, these receptors are generated by site-specific mutagenesis, sequenced for confirmation of the amino acid alteration, and screened for constitutive activity. Agonist induced signaling is included as a positive control; this also enables normalization/comparison of elevations in basal signaling (i.e. agonist induced signaling=100%).

In the alternative, random mutations may be introduced into a limited domain of the dopamine receptor of interest; mutant receptors are then screened for ligand independent signaling. Preferred domains for such mutagenesis include the amino and carboxy ends of the third intracellular loop as well as the sixth transmembrane domain.

As described above, mutants are screened with a series of luciferase reporter gene assays to detect Gs, Gi/o, and Gq mediated signaling. To confirm that Gs coupled mutants are constitutively active, basal cAMP production may be assessed using the flashplate assay (NEN). Agonist stimulated levels of cAMP or comparison with a known constitutively active Gs coupled receptor mutant (e.g., PTH receptor T410P) may be included as positive controls.

For dopamine receptor mutants that trigger Gi/o mediated signaling, confirmation of constitutive activity may be carried out in forskolin-stimulated cells. Basal signaling in forskolin treated cells expressing the wild type vs. constitutively active mutant are compared. The elevation in cAMP (or corresponding luciferase activity) resulting after forskolin stimulation should be decreased to a greater extent in cells expressing the constitutively active (vs. WT) receptors.

If the luciferase results suggest that constitutively active mutants are Gq coupled (i.e., activate the SRE-luciferase to a greater extent than the corresponding wild type receptor), follow up confirmatory studies may be used to assess the basal (i.e., ligand independent) level of receptor mediated production of inositol phosphates. Agonist stimulated levels of inositol phosphate production or comparison with a known constitutively active Gq coupled receptor mutant (e.g., CCK-2R, L325E) may be included as positive controls.

As a final test of constitutive activity, cells expressing constitutively active mutants may be treated with inverse agonists. Known inverse agonists for both the D1 and D2 receptors include (+)-butaclamol, haloperidol, and clozapine (Wilson et al., J. Neurochem. 77:493-504 (2001); Cai et al., Mol. Pharmacol. 56:989-996 (1999). These compounds inhibit ligand-independent signaling, and thus confirm mutation induced receptor activation.

To confirm the efficacy of constitutively active dopamine receptors, in vivo function of such receptors in adult rats may also be characterized. Specifically, recombinant adeno-associated viral constructs encoding the constitutively active receptors are injected unilaterally into rat striatum and ‘circling behavior’ quantified as an index of mutant receptor efficacy. It has previously been established that asymmetric striatal dopamine receptor mediated signaling results in circling behavior, away from the side with increased receptor mediated signaling. In animal models with unilateral overexpression of wild type D2 receptors resulting from infection with the corresponding adenoviral construct (Ikari et al., Brain Res. Mol. Brain Res. 34:315-320 (1995); Ingram et al., Exp. Gerontol. 33:793-804 (1998), peripheral administration of apomorphine (a dopamine receptor agonist) results in circling. Asymmetry in striatal dopamine 2 receptor expression has also been achieved by unilateral administration of 6-hydroxydopamine (6-OHDA), a neurotoxin that destroys nigrostriatal neurons and leads to an upregulation of D2 receptors on the 6-OHDA injected side (Sibley, Annu. Rev. Pharmacol. Toxicol. 39:313-341 (1999); Ozawa et al., J. Neural Transm. Suppl. 58:181-191 (2000); Ungerstedt et al., Brain Res. 24:485-493 (1970); Mendez et al., J. Neurosurg 42:166-173 (1975). Again, peripherally administered apomorphine results in circling behavior away from the side of increased receptor activity.

Because unilateral expression of constitutively active mutant dopamine receptors in the striatum is expected to result in asymmetric receptor mediated signaling, over-expression of such receptors should induce circling behavior independent of agonist stimulation. Without being bound to a particular theory (Emilien et al., Pharmacol. Ther. 84:133-156 (1999); Missale et al., Physiol. Rev. 78:189-225 (1998); Paul et al., J. Neurosci. 12:3729-3742 (1992); Usiello et al., Nature 408:199-203 (2000); Sibley, Annu. Rev. Pharmacol. Toxicol. 39:313-341 (1999), we believe the best candidate receptors to induce locomotor activity are the constitutively active D2L receptors, expressed either alone or in combination with constitutively active D1 receptors.

Dopamine Receptor Constructs

Complementary DNAs encoding each of the wild type and mutant D1, D2L, and D2S receptors are cloned into a rAAV transfer plasmid. This construct includes a neuron specific enolase promoter and an internal ribosomal entry site driving receptor and, for animal tests, green fluorescent protein expression bicistronically (Klein et al., Brain Res. 847:314-320 (1999). Co-expression of green fluorescent protein allows rapid assessment of transduction efficiency. Similar rAAV constructs have been demonstrated to give high-level striatal expression. Any rAAV construct may be used in the methods of the invention, for example, those rAAV constructs available from the University of Florida's Gene Therapy Center (Vector Core Facility) (see, for example, http://www.gtc.ufl.edu/gtc-home.htm; http://www.gtc.ufl.edu/gtc-vraav.htm).

Recombinant AAV provides a number of advantages (Ozawa et al., J. Neural. Transm. Suppl. 58:181-191 (2000); Bjorklund et al., Brain Res. 886:82-98 (2000); Mandel et al., Experimental Neurology 159:47-64 (1999). First, the wild type vector lacks any disease association. Second, rAAV can be used with transcripts up to 5 Kb; dopamine receptor transcripts are ˜1.5-2 Kb. Third, transgenes integrate into the host genome resulting in stable expression. Fourth, immune response to rAAV is markedly diminished since 96% of the viral genome has been removed; only genes for packaging and integration remain intact. Fifth, rAAV can transduce both non-dividing and dividing cells. Sixth, well-documented, high efficiency transduction occurs in striatal neurons. And, seventh, high-level expression is achieved for at least 2-6 months post infection.

For each dopamine receptor, virus encoding wild type and a constitutively active mutant (ideally with 50-100% activity, relative to the dopamine induced maximum, as assessed by in vitro assays) are generated. An empty rAAV vector is utilized as an additional negative control.

As each preparation of rAAV is completed, constructs are tested in HEK293 cells to ensure adequate receptor expression as well as confirmation of basal receptor mediated signaling. After rAAV infection, receptor densities are determined using homologous competition binding experiments with tritiated SCH 23390 or tritiated spiperone, selective radioligands for the D1 or D2 receptor, respectively Ozawa et al., J. Neural. Transm. Suppl. 58:181-191 (2000); Ingram et al., Mech. Ageing Dev. 116:77-93 (2000). Constitutive activity is verified with the appropriate luciferase reporter assay, SMS-luciferase for the D1 receptor and SRE-luciferase/Gq5i for the D2 receptor.

Constructs (rAAV encoding a constitutively active mutant receptor, a wild type receptor, or no receptor) may then be tested in male Sprague-Dawley rats (250-300 g) of comparable age for effects on circling behavior as described above. Ten animals will comprise each group. In these tests, each rat receives a single unilateral injection of rAAV, 4 μl of a˜10¹² particles per ml stock, into the dorsolateral striatum (DLS). This dose of virus is similar to ones used in earlier studies that successfully targeted the striatum (Ozawa et al., J. Neural. Transm. Suppl. 58:181-191 (2000); Bjorklund et al., Brain Res. 886:82-98 (2000); Klein et al., Brain Res. 847:314-320 (1999). A rAAV construct encoding GFP may be used to confirm that the striatal coordinates for injection (as per the Paxinos and Watson, Stereotaxic Atlas of the Rat Brain, 1998) target the DLS. In these animals it may also be determined whether and to what extent there is expression of GFP outside the targeted region; appropriate adjustments in dose, number of injections, and/or coordinates may be made based on these measurements.

Circling behavior in ten adult male rats is compared with equal numbers of controls. Animals are evaluated every other day for the onset of circling behavior by placing rats in a circular chamber (diameter=36 cm.) and monitoring behavior. Circling is recorded and quantified using the Ethovision video monitoring system (Noldus Information Technologies, Sterling, Va.). If no spontaneous circling behavior is evident after 5 weeks, animals are evaluated after peripheral administration of apomorphine, a dopamine receptor agonist. The 5-week interval allows ample time to achieve a stable level of receptor expression levels (Ozawa et al., J. Neural. Transm. Suppl. 58:181-191 (2000); Bjorklund et al., Brain Res. 886:82-98 (2000). Apomorphine-induced circling away from the side of the rAAV injection indicates that the viral construct induced receptor overexpression/asymmetry. At the same time, a lack of spontaneous circling in the absence of drug treatment suggests that the level of receptor expression and/or basal activity was not sufficient to induce spontaneous circling. In this case, expression levels may be increased by utilizing a higher dose of the injected rAAV construct and/or by widening the striatal field injected (Ozawa et al., J. Neural. Transm. Suppl. 58:181-191 (2000); Bjorklund et al., Brain Res. 886:82-98 (2000). As detailed below, the level of receptor expression is quantified by receptor autoradiography to monitor how alterations in dose/injection pattern influence striatal receptor density. Alternatively, the rAAV constructs may be further optimized by identifying additional point mutations that confer a greater degree of constitutive activity, as described above.

Once results are known with each construct individually, a combination of the constitutively active D2L and D1 rAAV constructs may be injected in parallel in equal amounts. A combination of corresponding wild type constructs are used as a control.

In addition to enhancing locomotor behavior, excess receptor activity might result in abnormal movements including writhing and/or tremors. In this case, a lower dose of the injected rAAV construct(s) is used and/or the striatal field injected is narrowed. Alternatively, the relevant rAAV construct(s) could be made with a less constitutively active receptor mutant.

Receptor expression is assessed in all rats (i.e., those that circle as well as those that do not) after completion of circling behavior studies. Rats are anesthetized with pentobarbital. The animals are then perfused transcardially with phosphate buffered saline followed by 4% paraformaldehyde w/sucrose. Brains are removed, frozen, and cut into transverse sections (20 microns) that extend through the striatum bilaterally. Since the rAAV constructs used in the animal tests encode green fluorescent protein (GFP) in parallel with the receptors, GFP expression provides a rapid index of protein expression. The brain sections also allow assessment of (i) tissue damage, (ii) accuracy of cannula placement, and (iii) dorsolateral striatum specific expression. To quantify striatal receptor expression, frozen brain sections are assessed using receptor autoradiography with subtype selective radioligands, tritiated spiperone for D2 receptors and tritiated SCH 23390 for D1 receptors (Sibley, D. R., Annu. Rev. Pharmacol. Toxicol. 39:313-341 (1999); Xu et al., Cell 79:729-742 (1994); Ingram et al., Mech. Ageing Dev. 116:77-93 (2000). The autoradiographic signals are measured using the Alpha Innotech Corp. ChemiImager 4400 densitometer. Parallel controls include animals injected with an empty rAAV as well as with rAAV encoding wild type receptors.

Constitutively active dopamine receptors may also be evaluated in other animal models of PD. rAAV constructs which result in spontaneous circling when expressed either alone (e.g. D2L CAM) or in combination (e.g. D2L-CAM+D1-CAM) maybe further evaluated using the 6 hydroxydopamine (6-OHDA) induced rat model of Parkinson's disease published by Diaz et al. (Rodriguez Diaz et al., Behav. Brain Res. 122:79-92 (2001); Breese, G. R., et al., Br. J. Pharmacol. 42:88-99 (1971); Rodriguez et al., Exp. Neurol. 169:163-181 (2001). In this model, 6-OHDA dose dependent decrease in spontaneous locomotor activity has been demonstrated with an accompanying increase in chewing behavior and catalepsy. Constitutively active receptors (vs. wild type receptors vs. empty rAAV construct) may be tested to determine whether their bilateral expression in the striatum protects against these 6-OHDA induced behavioral abnormalities. The protective effects of the constructs can be quantified relative to the dose of 6-OHDA administered.

Gene Therapy Using Constitutively Active Dopamine Receptor Nucleic Acid

Given the role of dopamine receptors in modulating locomotor function, constitutively active receptors provide a novel and useful approach to treating neurological disorders, such as Parkinson's disease. In this approach, nucleic acids encoding constitutively active dopamine receptors (for example, constitutively active dopamine 1 and/or dopamine 2 receptors) are administered, alone or in combination, to a mammal to increase dopaminergic activity. Preferably, these nucleic acids are delivered using recombinant adeno-associated viral vectors, and administration is preferably to the brain (for example, to the striatum).

In addition, as noted above, constitutively active dopamine receptors may be administered in conjunction with any other Parkinson's therapeutic including, without limitation, L-dopa, dopamine synthetic enzymes (for example, tyrosine hydroxylase or aromatic amino-dopacarboxylase), neuronal growth factors, or dopamine receptor agonists. Co-administration with the neuronal growth factor, glial cell line-derived neurotrophic factor (GNDF), represents a preferred co-administration approach.

Other Embodiments

The present invention provides therapeutic compositions that include nucleic acids encoding constitutively active, hypersensitive, or nonfunctional receptors and methods for delivering the therapeutic compositions to a mammal in need of treatment that may replace current agonist drug therapy. The skilled artisan will appreciate that any means of delivering the nucleic acid compositions to a cell, tissue, or mammal may be used in the present invention. One of ordinary skill in the art would also appreciate that the present invention is not limited to applications involving use of the G protein-coupled receptors, but may be extended to other constitutively active, hypersensitive, or nonfunctional receptors.

From the foregoing description, it will be apparent that variations and modifications may be made to the invention described herein to adopt it to various usages and conditions. Such embodiments are also within the scope of the following claims.

All publications mentioned in this specification are hereby incorporated by reference to the same extent as if each independent publication or patent application was specifically and individually indicated to be incorporated by reference.

What is claimed is:

1. A method of treating, reducing, or preventing pain in a mammal, said method comprising administering to said mammal a nucleic acid encoding a constitutively active mu opioid receptor in an amount sufficient to treat, reduce, or prevent pain.

2. The method of claim 1, wherein said mu opioid receptor has an single point mutation in transmembrane domain 3.

3. The method of claim 2, wherein said single point mutation is an Asn to Ala point mutation at amino acid 150 of SEQ ID NO: 1 or the human equivalent.

4. The method of claim 1, wherein said pain is back pain.

5. The method of claim 1, wherein the expression of said constitutively active mu opioid receptor is under the control of an inducible promoter.

6. The method of claim 1, wherein the expression of said constitutively active mu opioid receptor is under the control of a constitutive promoter.

7. The method of claim 1, wherein the expression of said constitutively active mu opioid receptor is under the control of a tissue specific promoter.

8. The method of claim 1, wherein said nucleic acid encoding said constitutively active mu opioid receptor is administered as part of a viral vector.

9. The method of claim 1, wherein said nucleic acid encoding said constitutively active mu opioid receptor is administered as part of a nonviral vector.

10. The method of claim 8 or 9, wherein said viral or nonviral vector includes cell specific ligands useful for targeting specific cell-types in a mammal.

11. The method of claim 8, wherein said viral vector is a retroviral or adenoviral vector.

12. The method of claim 8, wherein said viral vector is an adeno-associated viral vector.

13. A method of treating, reducing, or preventing pain in a mammal, said method comprising administering to said mammal a nucleic acid encoding a hypersensitive mu opioid receptor in an amount sufficient to treat, reduce, or prevent pain.

14. A therapeutic composition for treating, reducing, or preventing pain, comprising a nucleic acid encoding a constitutively active mu opioid receptor admixed with a pharmaceutically acceptable carrier substance, said nucleic acid being present in said composition in an amount equivalent to a unit dose suitable for administration to a mammal suffering from pain.

15. The therapeutic composition of claim 14, wherein said mu opioid receptor has a single point mutation in transmembrane domain 3.

16. The therapeutic composition of claim 15, wherein said single point mutation is a Asn to Ala point mutation at amino acid 150 of SEQ ID NO: 1.

17. The therapeutic composition of claim 14, wherein the expression of said constitutively active mu opioid receptor is under the control of an inducible promoter.

18. The therapeutic composition of claim 14, wherein the expression of said constitutively active mu opioid receptor is under the control of a constitutive promoter.

19. The therapeutic composition of claim 14, wherein the expression of said constitutively active mu opioid receptor is under the control of a tissue specific promoter.

20. The therapeutic composition of claim 14, wherein said nucleic acid encoding said constitutively active mu opioid receptor is administered as part of a viral vector.

21. The therapeutic composition of claim 20, wherein said viral vector is an adeno-associated viral vector.

22. The therapeutic composition of claim 14, wherein said nucleic acid encoding said constitutively active mu opioid receptor is administered as part of a nonviral vector.

23. The therapeutic composition of claim 20 or 22, wherein said viral or nonviral vector includes cell specific ligands useful for targeting specific cell-types in a mammal.

24. The therapeutic composition of claim 20, wherein said viral vector is a retroviral vector or adenoviral vector.

25. A therapeutic composition for treating, reducing, or preventing pain, comprising a nucleic acid encoding a hypersensitive mu opioid receptor admixed with a pharmaceutically acceptable carrier substance, said nucleic acid being present in said composition in an amount equivalent to a unit dose suitable for administration to a mammal suffering from pain.

26. A kit for the administration of a nucleic acid encoding a constitutively active mu opioid receptor to a mammal, comprising a container means containing a nucleic acid encoding a constitutively active mu opioid receptor in a pharmaceutically acceptable carrier.

27. The kit of claim 26, wherein said mu opioid receptor has a single point mutation in transmembrane domain 3.

28. The kit of claim 27, wherein said single point mutation is a Asn to Ala point mutation at amino acid 150 of SEQ ID NO: 1.

29. The kit of claim 26, wherein said nucleic acid is administered as part of a viral vector.

30. The kit of claim 29, wherein said nucleic acid is administered as part of an adeno-associated viral vector.

31. The kit of claim 26, wherein said nucleic acid is administered as part of a nonviral vector.

32. The kit of claim 29 or 31, wherein said viral or nonviral vector includes cell specific ligands useful for targeting specific cell-types in a mammal.

33. The kit of claim 29, wherein said viral vector is a retroviral vector or adenoviral vector.
